(19) United States
(12) Patent Application Publication
(10) Pub. No.: US 2018/0188254 A1
(43) Pub. Date: Jul. 5, 2018

(54) METHODS OF CHARACTERIZING ADRENOCORTICAL TUMORS

(71) Applicant: The United States of America, as Represented by the Secretary, Department of Health and Human serv, Bethesda, MD (US)

(72) Inventors: Dhaval T. Patel, Bethesda, MD (US); Kristopher W. Krausz, Bethesda, MD (US); Frank J. Gonzalez, Bethesda, MD (US); Electron Kebebew, Bethesda, MD (US); Matthew Thompson, Bethesda, MD (US)

(73) Assignee: The United States of America, as Represented by the Secretary, Department of Health and Human Serv, Bethesda, MD (US)

(21) Appl. No.: 15/740,577

(22) PCT Filed: Jun. 29, 2016
(86) PCT No.: PCT/US2016/040148

§ 371 Date: Dec. 28, 2017

Related U.S. Application Data
(60) Provisional application No. 62/186,218, filed on Jun. 29, 2015.

(51) Int. Cl. G01N 33/574; G01N 33/68

(52) U.S. Cl. CPC G01N 33/57438; G01N 33/6812

(57) ABSTRACT

Disclosed herein are methods of diagnosing and treating a malignant adrenocortical tumor, including adrenocortical carcinoma. In some examples, methods of diagnosing a malignant adrenocortical tumor include measuring creatine riboside, L-tryptophan, Nε,Nε,Nε-trimethyl-L-lysine and 3-methylhistidine in a biological sample obtained from a subject with an adrenocortical tumor and identifying an increase in creatine riboside and a decrease in L-tryptophan, Nε,Nε,Nε-trimethyl-L-lysine and 3-methylhistidine in the biological sample when compared to a control or reference value for each molecule indicates a malignant adrenocortical tumor. Methods of treatment and evaluating the effectiveness of an agent for treating a malignant adrenocortical tumor are also disclosed. Additionally, kits, assays and devices for characterizing adrenocortical tumors are provided.

### CROSS REFERENCE TO RELATED APPLICATION

This application claims priority to U.S. Provisional Application No. 62/186,218, filed Jun. 29, 2015, which is incorporated by reference in its entirety.

### FIELD

The present disclosure relates to the field of adrenal tumors and in particular, to non-invasive methods of characterizing adrenocortical tumors as benign or malignant.

### BACKGROUND

Adrenal neoplasms are common and incidentally detected in up to 14% of imaging studies. Because these tumors may be cancerous many patients undergo an operation to remove the gland, which accounts for approximately 50,000 operations a year in the United States. On pathology most of these tumors are found to be benign and in some cases a reliable diagnosis cannot be established to exclude adrenocortical carcinoma, and patients require continued follow up in these cases. Adrenocortical carcinoma, however, is rare with an annual incidence rate of 0.5-2 cases per million. Without clinical evidence of local invasion or distant metastasis a diagnosis of adrenocortical carcinoma cannot be excluded. Histologically, the Weiss scoring system is used for diagnosis, however, given the subjective nature of interpreting the Weiss histologic features some tumors are incorrectly diagnosed as benign and recur. Thus, the presently available diagnostic tools often fail to accurately diagnose adrenocortical tumors and result in unnecessary operations in the majority of patients with benign adrenal tumors.

### SUMMARY

Disclosed herein are metabolic markers that can accurately indicate adrenocortical carcinoma preoperatively and allow unnecessary operations in subjects with benign adrenal tumors to be avoided. In particular, four metabolite markers have been discovered and identified in significantly discriminating between benign and malignant adrenal neoplasms: creatine riboside, L-tryptophan, Nε,Nε,Nε-trimethyl-L-lysine, and 3-methylhistidine. The calculated areas under the curve for each metabolite was 0.79 for creatine riboside, 0.86 for L-tryptophan, 0.82 for Nε,Nε,Nε-trimethyl-L-lysine, and 0.78 for 3-methylhistidine. In combination, the four markers have an area under the curve of 0.89 and an accuracy of 79% in the diagnosis of adrenocortical carcinoma. Cross validation analysis showed that these four markers have a sensitivity of 94.7%, specificity of 82.6%, and positive predictive value of 69.2% and a negative predictive value of 97.4%.

Based on these findings, methods of characterizing an adrenocortical tumor are provided. Also provided are methods of diagnosing an adrenocortical tumor. Further provided are methods of treating a malignant adrenocortical tumor in a subject. Methods of determining the effectiveness of an agent for the treatment of a malignant adrenocortical tumor in a subject with the malignant adrenocortical tumor are disclosed. Moreover, assays, kits and devices are provided for detecting creatine riboside, L-tryptophan, Nε,Nε,Nε-trimethyl-L-lysine, and 3-methylhistidine which can be used to distinguish between a benign and malignant adrenocortical carcinoma.

The foregoing and other features of the disclosure will become more apparent from the following detailed description, which proceeds with reference to the accompanying figures.

### BRIEF DESCRIPTION OF THE DRAWINGS

FIG. 1 is a flowchart illustrating an exemplary approach to identifying potentially discriminating metabolites between adrenocortical carcinoma (ACC) and benign adrenal tumors.

FIGS. 2A and 2B provide PCA plots showing discrimination between patients with ACC (open circles) and benign (closed circles) adrenal disease in reverse phase (RP) in both (FIG. 2A) ESI+ and (FIG. 2B) ESI− mode.

FIGS. 2C and 2D provide supervised OPLS-DA plots showing differences between patients with ACC (open circles) and benign (closed circles) adrenal disease in both (FIG. 2C) ESI+ and (FIG. 2D) ESI-mode.

FIGS. 2E and 2F provide S-Plot correlative analyses showing significant variables correlated with patients with ACC or benign adrenal disease in both (FIG. 2E) ESI+ and (FIG. 2F) ESI− mode.

FIGS. 3A and 3B provide PCA plots showing discrimination between patients with ACC (open circles) and benign (closed circles) adrenal disease in HILIC phase in both (FIG. 3A) ESI+ and (FIG. 3B) ESI-mode.

FIGS. 3C and 3D provide supervised OPLS-DA plots showing differences between patients with ACC (open circles) and benign (closed circles) adrenal disease in both (FIG. 3C) ESI+ and (FIG. 3D) ESI-mode.

FIGS. 3E and 3F provide S-Plot correlative analyses showing significant variables correlated with patients with ACC or benign adrenal disease in both (FIG. 3E) ESI+ and (FIG. 3F) ESI− mode.

FIG. 4A is a multivariate Receiver Operating Characteristic (ROC) curve analysis showing improving area under the curve (AUC) with a combination of significant features.

FIG. 4B illustrates the predictive ability of the features to differentiate between patients with ACC (open circles) and patients with benign adrenal disease (filled circles).

FIG. 4C illustrates the predictive accuracy of the combined features (84%).

FIGS. 5A-5H provide a comparison and ROC curve analysis between patients with ACC and benign adrenal disease with respect to the four identified biomarkers: (FIGS. 5A, 5B) creatine riboside, (FIGS. 5C, 5D) L-tryptophan, (FIGS. 5E, 5F) Nε,Nε,Nε-trimethyl-L-lysine and (FIGS. 5G, 5H) 3-methylhistidine.

FIG. 6A is a multivariate ROC curve analysis showing improving AUC with a combination of creatine riboside, L-tryptophan, Nε,Nε,Nε-trimethyl-L-lysine, and 3-methylhistidine.

FIG. 6B provides the predictive ability of the features to differentiate between patients with ACC (filled circles) and patients with benign adrenal disease (open circles).

FIG. 6C provides the predictive accuracy of the combined features (79.5%).

FIGS. 7A and 7B provide PCA plots showing discrimination between patients with mitotane ingestion (open circles) and without mitotane ingestion (closed circles) in reverse phase (RP) in both (FIG. 7A) ESI+ and (FIG. 7B) ESI− mode.

FIGS. 7C and 7D provide supervised OPLS-DA plots showing differences between patients with mitotane ingestion (open circles) and without mitotane ingestion (closed circles) in both (FIG. 7C) ESI+ and (FIG. 7D) ESI− mode.

FIGS. 7E and 7F provide S-plot correlative analysis showing significant variables not correlated with patients with mitotane ingestion in both (FIG. 7E) ESI+ and (FIG. 7F) ESI− mode.

FIGS. 8A and 8B provide PCA plots showing discrimination between patients with mitotane ingestion (open circles) and without mitotane ingestion (closed circles) in HILIC in both (FIG. 8A) ESI+ and (FIG. 8B) ESI− mode.

FIGS. 8C and 8D provide supervised OPLS-DA plots showing differences between patients with mitotane ingestion (open circles) and without mitotane ingestion (closed circles) in both (FIG. 8C) ESI+ and (FIG. 8D) ESI− mode.

FIGS. 8E and 8F provide S-plot correlative analysis showing significant variables not correlated with patients with mitotane ingestion in both (FIG. 8E) ESI+ and (FIG. 8F) ESI− mode.

FIG. 9 provides MS-MS confirmation of identity of creatine riboside in comparison to commercial available and synthesized standards.

FIG. 10 provides MS-MS confirmation of identity of L-tryptophan in comparison to commercial available and synthesized standards.

FIG. 11 provides MS-MS confirmation of identity of Nε,Nε,Nε-trimethyl-L-lysine in comparison to commercial available and synthesized standards.

FIG. 12 provides MS-MS confirmation of identity of 3-methylhistidine in comparison to commercial available and synthesized standards.

FIGS. 13A-13D provide comparison of patients with ACC and benign adrenal disease in an independent set of samples. Levels were quantified for (FIG. 13A) creatine riboside, (FIG. 13B) L-tryptophan, (FIG. 13C) Nε,Nε,Nε-trimethyl-L-lysine, and (FIG. 13D) 3-methylhistidine. Open circles indicate patients not fasting.

### DETAILED DESCRIPTION

### I. Introduction

Adrenal masses detected incidentally by computed tomography (CT) scan are highly prevalent with a range from 4 to 10%. Management is guided by tumor size and growth, patient and imaging characteristics, and biochemical findings. Although these characteristics may help differentiate benign adrenal incidentalomas from the poor prognostic and rare adrenocortical carcinoma (ACC), patients frequently undergo adrenalectomy to exclude a cancer diagnosis. Thus, diagnostic biomarkers to distinguish benign from malignant adrenal neoplasm are needed.

In recent years, studies have focused on identifying diagnostic markers. One of the most promising markers has been microRNAs, which are ˜22 nucleotide, short stranded RNAs. It has been shown that miR-485-5p and miR-34a were elevated in patients with ACC compared to patients with benign adrenal disease. ACCs can be hormonally active, thus targeted urine metabolomic approaches using gas chromatography mass spectrometry has been utilized to differentiate patients with benign adrenal tumors and patients with ACC. This approach was able to discriminate between patients with benign adrenal tumors and ACC with a sensitivity and specificity of 90%.

Metabolomic analysis of body fluid samples focuses on identifying metabolites related to tumor biology. The two major metabolomic analytical tools used are nuclear magnetic resonance (NMR) and mass spectrometry (MS) coupled to a separation technique. Unlike NMR, MS provides semi-quantitative data with high sensitivity and the ability to identify low abundance metabolites. Liquid chromatography (LC) MS provides high-throughput analysis and reliable data quality. High-throughput LC/MS analysis of serum and urine samples is thus a platform that was used herein to discover new biomarkers for ACC. The inventors performed an untargeted metabolomic analysis with LCMS to identify a specific urinary metabolomic signature that can discriminate patients with benign adrenal tumors and patients with malignant adrenocortical tumors.

### II. Terms

Unless otherwise noted, technical terms are used according to conventional usage. Definitions of common terms in molecular biology may be found in Benjamin Lewin, Genes IX, published by Jones and Bartlett Publishers, 2007 (ISBN 0763740632); Kendrew et al. (eds.), The Encyclopedia of Molecular Biology, published by Blackwell Science Inc., 1998; and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

3-methylhistidine: A compound with the chemical name (25)-2-amino-3-(3-methylimidazol-4-yl), Chemical Abstracts Service (CAS) Registry Number 368-16-1, molecular weight of 169.18 and molecular formula C₇H₁₁N₃O₂. 3-methylhistidine has the chemical structure set forth as:

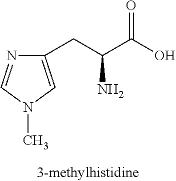

Administration: To provide or give a subject an agent, such as a therapeutic agent, by any effective route. Exemplary routes of administration include, but are not limited to, injection (such as subcutaneous, intramuscular, intradermal, intraperitoneal, intratumoral, and intravenous), oral, intraductal, sublingual, rectal, transdermal, intranasal, and inhalation routes.

Adrenocortical carcinoma (ACC): A rare but aggressive malignancy of the adrenal cortex. Adrenocortical carcinoma (also called “adrenal carcinoma”) is cancer that affects 1 to 2 people per million per year and accounts for 0.02-0.2% of all cancer deaths. Approximately half of all patients have metastatic disease at the time of diagnosis resulting in an average five-year survival of less than 10%. Currently there is limited knowledge regarding the initiation and pathophysiology of ACC.

Metastatic disease or local invasion is the only absolute indicator of malignancy. Masses without these features are assessed preoperatively based on size and imaging characteristics, although the findings of these studies often are unable to definitively categorize the tumor as benign or malignant. After resection, tumor pathology is assessed based on several histologic criteria including cell morphology, cellular proliferation, and tumor invasiveness (Weiss criteria). The only curative treatment is complete surgical excision of the tumor, which can be performed even in the case of invasion into large blood vessels, such as the renal vein or inferior vena cava. A large percentage of patients are not surgical candidates. Radiation therapy and radiofrequency ablation may be used for palliation in patients who are not surgical candidates.

Chemotherapy regimens typically include mitotane, an inhibitor of steroid synthesis which is toxic to cells of the adrenal cortex, as well as standard cytotoxic drugs. One widely used regimen consists of cisplatin, doxorubicin, etoposide, and mitotane. The endocrine cell toxin streptozotocin has also been included in some treatment protocols. Chemotherapy may be given to patients with unresectable disease, to shrink the tumor prior to surgery (neoadjuvant chemotherapy), or in an attempt to eliminate microscopic residual disease after surgery (adjuvant chemotherapy). Hormonal therapy with steroid synthesis inhibitors such as aminoglutethimide may be used in a palliative manner to reduce the symptoms of hormonal syndromes. In some examples, the disclosed methods include administration of such chemotherapy agents.

Agent: Any protein, nucleic acid molecule (including chemically modified nucleic acids), compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject, including inhibiting or treating a malignant adrenocortical tumor, such as inhibiting or treating ACC). For example, a “therapeutic agent” is a chemical compound, small molecule, or other composition, such as an antisense compound, antibody, protease inhibitor, hormone, chemokine or cytokine, capable of inducing a desired therapeutic or prophylactic effect when properly administered to a subject. In some examples, the therapeutic agent includes a metabolite that is down-regulated in patients with ACC or an inhibitor of a metabolite that is up-regulated in patients with ACC.

Alteration in metabolite level: An alteration in metabolic level of a metabolite refers to a change or difference, such as an increase or decrease, in the level of a metabolite, that is detectable in a biological sample (such as a urine sample) relative to a control or reference value of the metabolite in a subject with a benign adrenocortical tumor or from a subject without an adrenocortical tumor, such as an increase of at least 10%, at least 20%, at least 30%, at least 40%, at least 50%, at least 60%, at least 70%, at least 80%, at least 90%, at least 95%, at least 100%, at least 200%, at least 300%, at least 40% or at least 500%, or a decease of at least 10%, at least 20%, at least 30%, at least 40%, at least 50%, at least 60%, at least 70%, at least 80%, at least 90%, at least 95%, at least 98%, or at least 99%. An “alteration” in metabolite level includes an increase or a decrease in metabolite level. In some examples, the difference is relative to a control or reference value, such as an amount of metabolite in a sample from a healthy control subject or a benign adrenocortical tumor.

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called DNA chips or biochips. Protein-based arrays include probe molecules that are or include proteins, or where the target molecules are or include proteins. In some examples, an array contains antibodies to metabolites, such as the disclosed malignant adrenocortical metabolites, such as any combination of (such as at least 4 metabolites), including creatine riboside, L-Tryptophan, Nε,Nε,Nε-trimethyl-L-lysine and 3-methylhistidine. In some examples, the array consists of antibodies specific for creatine riboside, L-Tryptophan, Nε,Nε,Nε-trimethyl-L-lysine and 3-methylhistidine.

The array of molecules (“features”) makes it possible to carry out a very large number of analyses on a sample at one time. In certain example arrays, one or more molecules (such as an antibody or peptide) will occur on the array a plurality of times (such as twice), for instance to provide internal controls. The number of addressable locations on the array can vary, for example from at least one, to at least 2, to at least 3, at least 4, at least 5, at least 6, at least 10, at least 20, at least 30, at least 50, at least 75, at least 100, at least 150, at least 200, at least 300, at least 500, least 550, at least 600, at least 800, at least 1000, at least 10,000, or more. In some examples, arrays include positive and/or negative controls, such as housekeeping markers.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Biological sample: A biological specimen containing genomic DNA, RNA (including mRNA and microRNA), protein, or combinations thereof, obtained from a subject. Examples include, but are not limited to, saliva, peripheral blood, urine, tissue biopsy, fine needle aspirate, surgical specimen, and autopsy material. In one embodiment, the biological sample is a urine sample. In other embodiments, the biological sample is blood, or a component thereof, such as plasma or serum.

Chemotherapeutic agents: Any chemical agent with therapeutic usefulness in the treatment of diseases characterized by abnormal cell growth. Such diseases include tumors, neoplasms, and cancer, including ACC. In some cases, a chemotherapeutic agent is a radioactive compound. One of skill in the art can readily identify a chemotherapeutic agent of use (e.g., see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer, L., Berkery, R. (eds): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Combination chemotherapy is the administration of more than one agent to treat cancer.

Contacting: Placement in direct physical association, including both a solid and liquid form. Contacting an agent with a cell can occur in vitro by adding the agent to isolated cells or in vivo by administering the agent to a subject.

Control: A “control” refers to a sample or standard used for comparison with a test sample, such as a biological sample obtained from a patient (or plurality of patients) with a benign adrenocortical tumor. In some embodiments, the control is a sample obtained from a healthy patient (or plurality of patients) (also referred to herein as a “normal” control), such as a normal adrenocortical sample. In some embodiments, the control is a historical control or standard value (e.g., a previously tested control sample or group of samples that represent baseline or normal values, such as baseline or normal values in a benign adrenocortical tumor). In some examples the control is a standard value representing the average value (or average range of values) obtained from a plurality of patient samples (such as an average value or range of values of a metabolite from normal patients (patients that do not have an adrenocortical tumor) or an average level in patients with benign adrenocortical tumor samples).

Creatine riboside: A compound with the chemical name 2-{2-[(2R,3R,4S,5R)-3,4-dihydroxy-5-(hydroxymethyl)-oxolan-2-yl]-1-methylcarbamimidamido}acetic acid, Chemical Abstracts Service (CAS) Registry Number 1616693-92-5, and molecular weight of 263.25. Creatine riboside has the chemical structure set forth as:

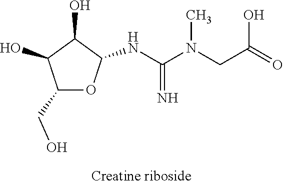 [SMILES: CN(CC(=O)O)C(=N)N[C@@H]1O[C@H](CO)[C@@H](O)[C@H]1O]

Decrease: To reduce the quality, amount, or strength of something. In one example, a metabolite level is decreased in a subject with ACC as compared to a subject with a benign tumor or a healthy subject. For example, L-Tryptophan, Nε,Nε,Nε-trimethyl-L-lysine and 3-methylhistidine were each lower/decreased in patients with ACC compared to patients with benign adrenal tumors. In other examples, a therapy decreases a tumor (such as the size of a tumor, the number of tumors, the metastasis of a tumor, or combinations thereof), or one or more symptoms associated with a tumor, for example as compared to the response in the absence of the therapy. In a particular example, a therapy decreases the size of a tumor, the number of tumors, the metastasis of a tumor, or combinations thereof, subsequent to the therapy, such as a decrease of at least 10%, at least 20%, at least 50%, or even at least 90%. Such decreases can be measured using the methods disclosed herein. In some examples, when used in reference to decreased/lower metabolite levels, a reduction or decrease refers to any process which results in a decrease in production of the metabolite. In certain examples, a decrease in the production/level of a metabolite is by at least 2-fold, at least 3-fold, at least 4-fold, at least 5-fold, at least 6-fold, at least 8-fold, at least 10-fold, at least 15-fold, at least 20-fold, at least 30-fold or at least 40-fold, as compared to a control (such as to a subject with a benign adrenal tumor).

Detect: To determine if a particular agent is present or absent, and in some example further includes quantification of the agent if detected.

Diagnosis: The process of identifying a disease by its signs, symptoms and/or results of various tests. The conclusion reached through that process is also called “a diagnosis.” Forms of testing commonly performed include blood tests, medical imaging, genetic analysis, urinalysis, and biopsy.

Diagnostically significant amount: As used herein a “diagnostically significant amount” refers to an increase or decrease in the level of a metabolite in a biological sample that is sufficient to allow one to distinguish one patient population from another (such as a malignant adrenocortical tumor from a benign adrenocortical tumor). In some embodiments, the diagnostically significant increase or decrease is at least 2-fold, at least 3-fold, at least 4-fold, at least 5-fold, at least 6-fold, at least 8-fold, at least 10-fold, at least 15-fold, at least 20-fold, at least 30-fold or at least 40-fold relative to a control. In some examples, the diagnostically significant amount is the level-change in metabolites shown in the Examples Section, such as at least a 1.5-fold change, such as a 2-fold change in creatine riboside, L-Tryptophan, Nε,Nε,Nε-trimethyl-L-lysine and 3-methylhistidine (such as an at least 1.5-fold decrease in L-Tryptophan, Nε,Nε,Nε-trimethyl-L-lysine and 3-methylhistidine) or at least a 2-fold change in creatine riboside (such as an at least 2-fold increase in creatine riboside) relative to a control (such as a subject in with a benign adrenal tumor or a reference value indicative of the levels of such metabolites in a subject with a benign adrenal tumor). A diagnostically significant amount can also be determined by measuring metabolite levels by methods disclosed herein.

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease. In some examples, an “effective amount” is a therapeutically effective amount in which the agent alone with an additional therapeutic agent(s) (for example a chemotherapeutic agent), induces the desired response such as treatment of a tumor, such as a malignant adrenocortical tumor. In one example, a desired response is to decrease tumor and/or metastasis, size, volume, or number in a subject to whom the therapy is administered. Tumor metastasis does not need to be completely eliminated for the composition to be effective. For example, a composition can decrease tumor and/or metastasis, size, volume, or number by a desired amount, for example by at least 20%, at least 50%, at least 60%, at least 70%, at least 80%, at least 90%, at least 95%, at least 98%, or even at least 100% (elimination of the tumor), as compared to tumor and/or metastasis, size, volume, or number in the absence of the composition.

In particular examples, it is an amount of an agent effective to decrease a number of malignant adrenocortical carcinoma cells, such as in a subject to whom it is administered, for example a subject having one or more carcinomas. The cancer cells do not need to be completely eliminated for the composition to be effective. For example, a composition can decrease the number of cancer cells by a desired amount, for example by at least 20%, at least 50%, at least 60%, at least 70%, at least 80%, at least 90%, at least 95%, at least 98%, or even at least 100% (elimination of detectable cancer cells), as compared to the number of cancer cells in the absence of the composition.

In other examples, it is an amount of an agent capable of modulating one or more of the disclosed metabolites associated with a malignant adrenocortical tumor (such as associated with ACC) by least 20%, at least 50%, at least 60%, at least 70%, at least 80%, at least 90%, at least 95%, at least 98%, or even at least 100% by the agent.

Increase: To increase the quality, amount, or strength of something. In one example, a metabolite level is increased in a subject with ACC as compared to a subject with a benign tumor or a healthy subject. For example, creatine riboside is higher/increased in patients with ACC compared to patients with benign adrenal tumors. In certain examples, an increase in the production/level of a metabolite is by at least 2-fold, at least 3-fold, at least 4-fold, at least 5-fold, at least 6-fold, at least 8-fold, at least 10-fold, at least 15-fold, at least 20-fold, at least 30-fold or at least 40-fold, as compared to a control (such as to a subject with a benign adrenal tumor).

Label: An agent capable of detection, for example by ELISA, spectrophotometry, flow cytometry, or microscopy. For example, a label can be attached to a nucleic acid molecule, protein, metabolite, thereby permitting detection of such. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Lateral flow device: An analytical device in the form of a test strip used in lateral flow chromatography, in which a sample fluid, such as one to be tested for the presence of a target agent, flows (for example by capillary action) through the strip (which is frequently made of bibulous materials such as paper, nitrocellulose, and cellulose). The test sample and any suspended target agent(s) can flow along the strip to a detection zone in which glucose produced as a result of the presence or absence of the target agent is detected, to indicate a presence, absence and/or quantity of the target agent.

Numerous lateral flow analytical devices are known, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,368,876; 7,799,554; EP 0810436; and WO 92/12428; WO 94/01775; WO 95/16207; and WO 97/06439, each of which is incorporated by reference.

Lateral flow devices can in one example be a one-step lateral flow assay in which a sample fluid is placed in a sample or wicking area on a bibulous strip (though, non-bibulous materials can be used, and rendered bibulous by applying a surfactant to the material), and allowed to migrate along the strip until the sample comes into contact with one or more reagents.

In some examples, the strip includes multiple regions for detecting different test agents in the sample (for example in parallel lines or as other separate portions of the device). The test strips can also incorporate control indicators, which provide a signal that the test has adequately been performed, even if a positive signal indicating the presence (or absence) of a target is not achieved.

A lateral flow device can include a sample application area or wicking pad, which is where the fluid or liquid sample is introduced. In one example, the sample may be introduced to the sample application area by external application, as with a dropper or other applicator. In another example, the sample application area may be directly immersed in the sample, such as when a test strip is dipped into a container holding a sample. In yet another example, the sample may be applied, blotted, poured or expressed onto the sample application area.

A lateral flow device can include a reagent or conjugation pad, the region of a lateral flow device where reagents are immobilized, such as the starting products, conjugated antibodies, target-specific binding agent-bound solid substrates (such as antibody or aptamer immobilized to magnetic beads), target-bound solid substrates (such as targets immobilized to magnetic beads, Target-MB), antibodies specific for a target agent, or combinations thereof. A lateral flow device may have more than one conjugation area, for example, a “primary conjugation area,” a “secondary conjugation area,” and so on. Often different reagents are immobilized in the primary, secondary, or other conjugation areas. Multiple conjugation areas may have any orientation with respect to each other on the lateral flow substrate; for example, a primary conjugation area may be distal or proximal to a secondary (or other) conjugation area and vice versa. Alternatively, a primary conjugation area and a conjugation (or other) area may be oriented perpendicularly to each other such that the two (or more) conjugation areas form a cross or a plus sign or other symbol. For example, Apilux et al. (Anal. Chem. 82:1727-32, 2010), Dungchai et al. (Anal. Chem. 81:5821-6, 2009), and Dungchai et al. (Analytica Chemica Acta 674:227-33, 2010), provide exemplary lateral flow devices with a central sample area and one or more conjugation areas distal to the sample area, which provide independent test zones where independent reactions can occur (e.g., each test zone has a different reagents for detecting a particular test agent, and can further include one or more reaction pads where reactions can take place (for example interspersed between the reagent pads) and an absorption pad that receives the generated product which can then be read.

A lateral flow device can include one or more reaction pads, such as a membrane, that can be a place to allow desired reactions to occur, and an absorption pad that draws the sample across the conjugation pad(s) and membrane(s) by capillary action and collects it.

L-tryptophan: One of the standard amino acids with the chemical name tryptophan, (2S)-2-amino-3-(1H-indol-3-yl)propanoic acid or 2-Amino-3-(1H-indol-3-yl)propanoic acid, Chemical formula C₁₁H₁₂N₂O₂, and Chemical Abstracts Service (CAS) Registry Number 73-22-3. L-tryptophan has the chemical structure set forth as:

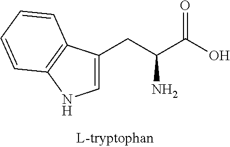 [SMILES: N[C@@H](Cc1c[nH]c2ccccc12)C(=O)O]

Mass spectrometry: A method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation.

Measuring the level of metabolite: As used herein, measuring the level of a particular metabolite refers to quantifying the amount of a metabolite, such as a urinary metabolite in a sample. Quantification can be either numerical or relative. Detecting the level of a particular metabolite can be achieved using any method known in the art or described herein.

In primary embodiments, the change detected is an increase or decrease in expression as compared to a control, such as a reference value (e.g., a value representing that found in a subject with a benign adrenal tumor or a healthy control subject) or a control. In some examples, the detected increase or decrease is an increase or decrease of at least two-fold compared with the control or standard. Controls or standards for comparison to a sample, for the determination of differential levels, include samples believed to be normal (in that they are not altered for the desired characteristic, for example a sample from a subject who does not have cancer, such as ACC, or a benign adrenal tumor) as well as laboratory values (e.g., range of values), even though possibly arbitrarily set, keeping in mind that such values can vary from laboratory to laboratory.

Laboratory standards and values can be set based on a known or determined population value and can be supplied in the format of a graph or table that permits comparison of measured, experimentally determined values.

In other examples, the detected increase or decrease is a change rounded down to the nearest whole number (so that both 2.05 and 2.67 are rounded down to 2) of the fold change shown for a metabolite in the Example Section, or is rounded to the nearest whole number (so that 2.05 would be rounded to 2 and 2.67 would be rounded to 3). In other embodiments of the methods, the increase or decrease is of a diagnostically significant amount, which refers to a change of a sufficient magnitude to provide a statistical probability of the diagnosis.

Nε,Nε,Nε-trimethyl-L-lysine: A compound with the chemical name N6,N6,N6-Trimethyl-L-lysine, molecular formula of C₉H₂₀N₂O₂, and is an α-amino-acid cation that is the N⁶-trimethyl derivative of L-lysine. The Chemical Abstracts Service (CAS) Registry Number is 19253-88-4. Nε,Nε,Nε-trimethyl-L-lysine has the chemical structure set forth as:

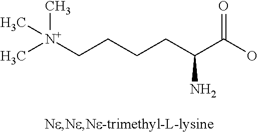

Metabolomics: The scientific study of chemical processes involving metabolites. Specifically, metabolomics is the systematic study of the unique chemical fingerprints that specific cellular processes leave behind, and the study of their small-molecule metabolite profiles.

Patient or Subject: A term that includes human and non-human animals, such as those having an adrenocortical tumor. In one example, the patient or subject is a mammal, such as a human. “Patient” and “subject” are used interchangeably herein.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds, molecules or agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Prognosis: A prediction of the course of a disease, such as cancer (for example, ACC). The prediction can include determining the likelihood of a subject to develop aggressive, recurrent disease, to develop one or more metastasis, to survive a particular amount of time (e.g., determine the likelihood that a subject will survive 1, 2, 3 or 5 years), to respond to a particular therapy or combinations thereof.

Tissue: A plurality of functionally related cells. A tissue can be a suspension, a semi-solid, or solid. Tissue includes cells collected from a subject, such as from the adrenal cortex. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Sensitivity: A measurement of activity, such as biological activity, of a molecule or collection of molecules in a given condition. In an example, sensitivity refers to the activity of any tumor molecule in the presence of therapeutic agent, such as an agent that targets one or more malignant tumor metabolites. In other examples, sensitivity refers to the activity of an agent (such as a therapeutic agent) on the growth, development or progression of a disease, such as ACC. For example, a decreased sensitivity refers to a state in which a tumor is less responsive to a given therapeutic agent as compared to a tumor that is responsive to the treatment.

In certain examples, sensitivity or responsiveness can be assessed using any endpoint indicating a benefit to the subject, including, without limitation, (1) inhibition, to some extent, of tumor growth, including slowing down and complete growth arrest; (2) reduction in the number of tumor cells; (3) reduction in tumor size; (4) inhibition (such as reduction, slowing down or complete stopping) of tumor cell infiltration into adjacent peripheral organs and/or tissues; (5) inhibition (such as reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

In some examples, sensitivity of an assay describes the ability of the assay to accurately predict whether one has a malignant adrenocortical tumor using the disclosed metabolites as compared to another assay method. For example, a metabolite with a sensitivity of at least 70%, at least 75%, at least 80%, at least 90%, at least 95% or greater sensitivity is one that is capable of accurately predicting a malignant adrenocortical tumor.

In contrast, “specificity” refers to the ability of a molecule to detect a malignant adrenocortical tumor as compared to a benign tumor.

Treating a disease: A phrase referring to a therapeutic intervention that ameliorates a sign or symptom of a disease or pathological condition after it has begun to develop.

Tumor, neoplasia: The result of abnormal and uncontrolled growth of cells or tissue that results from excessive cell division. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” “Malignant cells” are those that have the properties of anaplasia invasion and metastasis.

Under conditions sufficient for: A phrase that is used to describe any environment that permits the desired activity. In one example, includes administering a test agent to an ACC or a subject having ACC sufficient to allow the desired activity. In particular examples, the desired activity is altering the activity, such as increasing or decreasing the level of a malignant adrenocortical metabolite.

Weiss criteria: A combination of the following nine criteria for distinguishing malignant adrenocortical tumors from benign adrenocortical tumors: nuclear grade III or IV; mitotic rate greater than 5/50 high-power fields; atypical mitoses; clear cells comprising 25% or less of the tumor; a diffuse architecture; microscopic necrosis; and invasion of venous, sinusoidal, and capsular structures. The presence of three or more of these features in a given tumor indicates malignant potential.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Methods

Described herein is the identification of urinary metabolites that have altered levels in patients with malignant adrenocortical tumors, such as patients with ACC, compared with patients with benign adrenocortical tumors or healthy control subjects. Using LCMS analysis, numerous molecules were identified that were increased or decreased, such as by at least 1.8-fold in patients with a malignant adrenocortical tumor relative to a control (e.g., a benign adrenocortical tumor). In particular, specific metabolites were identified to be increased in malignant adrenocortical tumor samples whereas others were decreased in such samples. In some examples, creatine riboside, L-tryptophan, Nε,Nε,Nε-trimethyl-L-lysine, and/or 3-methylhistidine levels are altered by at least 1.8-fold. In some examples, creatine riboside are increased by at least 2-fold in malignant adrenocortical tumor samples whereas L-tryptophan, Nε,Nε,Nε-trimethyl-L-lysine, and/or 3-methylhistidine were decreased by at least 1.8-fold as compared to subjects with a benign adrenocortical tumor. Thus, by measuring levels of one or more of the altered metabolites in a sample obtained from a subject, one can characterize an adrenocortical tumor as benign or malignant which can be used to diagnose a subject as having a malignant adrenocortical tumor, and more particularly ACC. The urinary metabolites identified as being presented at altered levels in a malignant adrenocortical tumor as compared to a control can serve as therapeutic targets for treating the malignant adrenocortical tumor (including ACC). Further, methods of determining the efficacy of an agent for treating a malignant adrenocortical tumor, such as for treating ACC, are also disclosed. Moreover, methods of screening potential therapeutic agents are provided.

### A. Methods of Characterizing a Malignant Adrenocortical Tumor

Provided herein is a method of characterizing an adrenocortical tumor by measuring the level of at least one metabolite, such as creatine riboside, L-tryptophan, Nε,Nε,Nε-trimethyl-L-lysine, and/or 3-methylhistidine, in a biological sample (e.e., a urine sample) obtained from a subject suspected of having a malignant adrenocortical tumor. An alteration in the level of the at least one metabolite in the biological sample of the subject relative to a control indicates the adrenocortical tumor is malignant. As described herein, an increase in the level of creatine riboside, a decrease in the level of L-tryptophan, a decrease in the level of Nε,Nε,Nε-trimethyl-L-lysine, a decrease in the level of 3-methylhistidine or a combination thereof, in a urine sample obtained from a subject with a adrenocortical tumor relative to a control (such as reference values indicative of the level of said metabolites in a subject with a benign adrenocortical tumor), indicates the adrenocortical tumor is malignant.

In some embodiments of the methods, the increase or decrease in the metabolite level is at least 1.5-fold, such as at least 1.8-fold, at least 2-fold, at least 3-fold, at least 4-fold, at least 5-fold, at least 10-fold, including about 1.8-fold, about 1.9-fold, about 2-fold, about 3-fold, about 4-fold, about 5-fold, about 6-fold, about 7-fold, about 8-fold, about 9-fold, about 10-fold, about 30-fold, and about 100-fold. In particular examples, creatine riboside is increased by at least 2-fold, Nε,Nε,Nε-trimethyl-L-lysine is decreased by at least 1.8-fold, L-tryptophan is decreased by at least 3-fold and 3-methylhistidine is decreased by at least 3-fold relative to control values (e.g., levels of the given metabolites in a benign adrenocortical tumor or a reference value known to be indicative of such levels in a benign adrenocortical tumor).

It is understood that the methods disclosed herein include measuring the level of any feature, combination or subcombination of metabolites including creatine riboside, Nε,Nε,Nε-trimethyl-L-lysine, L-tryptophan, and/or 3-methylhistidine. In some cases, the method includes measuring the level of creatine riboside. In some cases, the method includes measuring the level of Nε,Nε,Nε-trimethyl-L-lysine. In some cases, the method includes measuring the level of L-tryptophan. In some cases, the method includes measuring the level of 3-methylhistidine.

In some cases, the method includes measuring the level of one or more metabolites described herein and one or more substances known to be associated with ACC. Methods of detecting and measuring metabolites are described in detail below. In some examples, sensitive assays, such as LCMS-, ELISA-, chemoluminiscence- or fluorescence-based kits, are used to measure the desired metabolites. In some examples, it is desirable to use microarray analysis.

In some embodiments of the methods, the biological sample is a urine sample. In some examples, the biological sample is a blood sample. Thus, the method in some examples includes obtaining an appropriate sample from the subject so that the specific metabolite levels may be measured.

B. Methods of Diagnosing a Subject with a Malignant Adrenocortical Tumor

Provided herein is a method of diagnosing a subject as having a malignant adrenocortical tumor by measuring the level of at least one metabolite, such as creatine riboside, L-tryptophan, Nε,Nε,Nε-trimethyl-L-lysine, and/or 3-methylhistidine, in a biological sample (e.g., urine sample) obtained from a subject. An alteration in the level of the at least one metabolite in the biological sample of the subject relative to a control indicates the subject has a malignant adrenocortical tumor. As described herein, an increase in the level of creatine riboside, a decrease in the level of L-tryptophan, a decrease in the level of Nε,Nε,Nε-trimethyl-L-lysine, a decrease in the level of 3-methylhistidine or a combination thereof, in a biological sample obtained from a subject with a adrenocortical tumor relative to a control (such as reference values indicative of the level of said metabolites in a subject with a benign adrenocortical tumor), indicates the subject has a malignant adrenocortical tumor. In some embodiments, the increase or decrease in the level of the metabolite is of a diagnostically significant amount.

In some embodiments of the methods, the diagnostically significant increase or decrease in the metabolite level is at least 1.5-fold, such as at least 1.8-fold, at least 2-fold, at least 3-fold, at least 4-fold, at least 5-fold, at least 10-fold, including about 1.8-fold, about 1.9-fold, about 2-fold, about 3-fold, about 4-fold, about 5-fold, about 6-fold, about 7-fold, about 8-fold, about 9-fold, about 10-fold, about 30-fold, and about 100-fold. In particular examples, creatine riboside is increased by at least 2-fold, Nε,Nε,Nε-trimethyl-L-lysine is decreased by at least a 1.8-fold, L-tryptophan is decreased by at least 3-fold and 3-methylhistidine is decreased by at least 3-fold relative to control values (e.g., levels of the given metabolites in a benign adrenocortical tumor or a reference value known to be indicative of such levels in a benign adrenocortical tumor).

It is understood that the methods disclosed herein include measuring the level of any feature, combination or subcombination of features listed in Table 3.

In particular examples, the combination of metabolites includes creatine riboside, Nε,Nε,Nε-trimethyl-L-lysine, L-tryptophan, 3-methylhistidine or any subcombination thereof. In some cases, the method includes measuring the level of a single metabolite, such as one of creatine riboside, Nε,Nε,Nε-trimethyl-L-lysine, L-tryptophan, or 3-methylhistidine. In one example, the single metabolite is creatine riboside. In another example, the single metabolite is Nε,Nε,Nε-trimethyl-L-lysine. In another example, the single metabolite is L-tryptophan. In another example, the single metabolite is 3-methylhistidine

In some cases, the method includes measuring the level of one or more metabolites described herein and one or more substances known to be associated with ACC. Methods of detecting and measuring metabolites are described in detail below. In some examples, sensitive assays, such as LCMS-, ELISA-, chemoluminiscence- or fluorescence-based kits, are used to measure the desired metabolites. In some examples, it is desirable to use microarray analysis.

In some embodiments of the methods, the biological sample is a urine sample. Thus, the method in some examples includes obtaining an appropriate sample from the patient to be diagnosed or treated with the methods provided herein.

In some embodiments, the method further includes providing an appropriate therapy for the subject diagnosed with a malignant adrenocortical tumor, such as administration of one or more chemotherapeutic agents. In some examples, the therapy includes administering an agent that alters the level of ACC associated molecule, such as an agent that inhibits a metabolite identified as increased in a malignant adrenocortical tumor relative to a control, or s an agent that increases or agonizes a metabolite identified as decreased in a malignant adrenocortical tumor relative to a control.

In some embodiments, a patient with an adrenocortical tumor or suspected of having such, can be pre-selected for the treatment and screening methods herein.

In some embodiments, once a patient's diagnosis is determined, an indication of that diagnosis can be displayed and/or conveyed to a clinician or other caregiver. For example, the results of the test are provided to a user (such as a clinician or other health care worker, laboratory personnel, or patient) in a perceivable output that provides information about the results of the test. In some examples, the output is a paper output (for example, a written or printed output), a display on a screen, a graphical output (for example, a graph, chart, voltammetric trace, or other diagram), or an audible output.

In other examples, the output is a numerical value, such as an amount of creatine riboside, Nε,Nε,Nε-trimethyl-L-lysine, L-tryptophan, 3-methylhistidine in the sample or a relative amount of creatine riboside, Nε,Nε,Nε-trimethyl-L-lysine, L-tryptophan, 3-methylhistidine in the sample as compared to a control or reference value. In additional examples, the output is a graphical representation, for example, a graph that indicates the value (such as amount or relative amount) of creatine riboside, Nε,Nε,Nε-trimethyl-L-lysine, L-tryptophan, or 3-methylhistidine in the sample from the subject on a standard curve. In a particular example, the output (such as a graphical output) shows or provides a cut-off value or level that indicates the presence of a malignant adrenocortical tumor. In some examples, the output is communicated to the user, for example by providing an output via physical, audible, or electronic means (for example by mail, telephone, facsimile transmission, email, or communication to an electronic medical record).

The output can provide quantitative information (for example, an amount of creatine riboside, Nε,Nε,Nε-trimethyl-L-lysine, L-tryptophan, 3-methylhistidine relative to a control sample or value) or can provide qualitative information (for example, a diagnosis of ACC). In additional examples, the output can provide qualitative information regarding the relative amount of creatine riboside, Nε,Nε,Nε-trimethyl-L-lysine, L-tryptophan, or 3-methylhistidine in the sample, such as identifying an increase in creatine riboside, relative to a control, a decrease in Nε,Nε,Nε-trimethyl-L-lysine, L-tryptophan, and/or 3-methylhistidine relative to a control, or no change in creatine riboside, Nε,Nε,Nε-trimethyl-L-lysine, L-tryptophan, and/or 3-methylhistidine relative to a control.

In some examples, the output is accompanied by guidelines for interpreting the data, for example, numerical or other limits that indicate the presence or absence of metastasis. The guidelines need not specify whether metastasis is present or absent, although it may include such a diagnosis. The indicia in the output can, for example, include normal or abnormal ranges or a cutoff, which the recipient of the output may then use to interpret the results, for example, to arrive at a diagnosis, prognosis, or treatment plan. In other examples, the output can provide a recommended therapeutic regimen. In some examples, the test may include determination of other clinical information (such as determining the amount of one or more additional adrenocortical cancer biomarkers in the sample).

In some embodiments, the disclosed methods of diagnosis include one or more of the following depending on the patient's diagnosis: a) prescribing a treatment regimen for the patient if the patient's determined diagnosis is considered to be positive for a malignant adrenocortical tumor; b) not prescribing a treatment regimen for the patient if the patient's determined diagnosis is considered to be negative for a malignant adrenocortical tumor; c) administering a treatment to the patient if the patient's determined diagnosis is considered to be positive for a malignant adrenocortical tumor; and d) not administering a treatment regimen to the patient if the patient's determined diagnosis is considered to be negative for a malignant adrenocortical tumor. In an alternative embodiment, the method can include recommending one or more of a)-d).

In some embodiments, an increase in creatine riboside, and a decrease in the levels of Nε,Nε,Nε-trimethyl-L-lysine, L-tryptophan, and/or 3-methylhistidine relative to a control or reference value is correlated with a poor prognosis. For example, if metabolite level is compared to a level of a subject with a benign adrenocortical tumor or reference value (level of metabolite level known to be present in a subject with a benign adrenocortical tumor), an increase in creatine riboside level of about 1.5-fold, about 2-fold, about 2.5-fold, about 3-fold, about 4-fold, about 5-fold, about 7-fold or about 10-fold relative to the control sample/reference value and a decrease in Nε,Nε,Nε-trimethyl-L-lysine, L-tryptophan, and/or 3-methylhistidine levels of about 1.5-fold, about 2-fold, about 2.5-fold, about 3-fold, about 4-fold, about 5-fold, about 7-fold or about 10-fold relative to the control sample, indicates a poor prognosis. In some examples, an increase in creatine riboside of about 1.3-fold to about 4-fold, such as about 1.5-fold to 3.5-fold relative to the control sample and a decrease in Nε,Nε,Nε-trimethyl-L-lysine, L-tryptophan, and/or 3-methylhistidine levels of about 1.3-fold to about 4-fold, such as about 1.5-fold to 3.5-fold relative to the control sample indicates a poor prognosis.

Poor prognosis can refer to any negative clinical outcome, such as, but not limited to, a decrease in likelihood of survival (such as overall survival, relapse-free survival, or metastasis-free survival), a decrease in the time of survival (e.g., less than 5 years, or less than one year), an increase in the severity of disease, a decrease in response to therapy, an increase in tumor recurrence, an increase in metastasis, or the like. In particular examples, a poor prognosis is a decreased chance of survival (for example, a survival time of equal to or less than 60 months, such as 50 months, 40 months, 30 months, 20 months, 12 months, 6 months or 3 months from time of diagnosis or first treatment).

In other examples, no significant change in levels of creatine riboside, Nε,Nε,Nε-trimethyl-L-lysine, L-tryptophan, and/or 3-methylhistidine relative to the control or a reference value known to be indicative of levels in a benign adrenocortical tumor indicates a good prognosis (such as increased chance of survival, for example increased overall survival, relapse-free survival, or metastasis-free survival). In an example, an increased chance of survival includes a survival time of at least 50 months from time of diagnosis, such as 60 months, 80 months, 100 months, 120 months or 150 months from time of diagnosis or first treatment.

### C. Methods of Treating a Malignant Adrenocortical Tumor

Also provided herein is a method of treating a patient with a malignant adrenocortical tumor, including ACC, by administering to the patient an agent that inhibits elevation of creatine riboside or increases levels of Nε,Nε,Nε-trimethyl-L-lysine, L-tryptophan, and/or 3-methylhistidine in the patient with a malignant adrenocortical tumor relative to a control. The agent can be any compound, such as a nucleic acid molecule, polypeptide, small molecule or other compound that is capable of inhibiting and/or reducing elevation of creatine riboside or increasing levels of Nε,Nε,Nε-trimethyl-L-lysine, L-tryptophan, and/or 3-methylhistidine in a biological sample obtained from a subject with a malignant adrenocortical tumor. For example, an agent can increase or decrease the metabolite level by a desired amount, for example by at least about 1.5-fold, at least about 2-fold, at least about 2.5-fold, at least about 3-fold, at least about 4-fold, at least about 5-fold, at least about 7-fold, or at least about 10-fold relative to activity or expression in a control (for example, the relative amount of expression in the absence of treatment). A control can be any suitable control, such as a reference value. For example, the reference value (or values if more than one metabolite is measured) can be an historical value based on average levels of the metabolite in a benign adrenocortical tumor or a healthy subject (a subject that has not been diagnosed with a malignant adrenocortical tumor, including ACC).

Treatment of ACC by altering the level of one or more of the disclosed metabolites associated with a malignant adrenocortical tumor (such as decreasing the level of creatine riboside by a desired amount, such as at least 10%, at least 20%, at least 50%, at least 70% or even at least 90% and increasing the level of Nε,Nε,Nε-trimethyl-L-lysine, L-tryptophan, and/or 3-methylhistidine by a desired amount, such as at least 10%, at least 20%, at least 50%, at least 70% or even at least 90%) can include delaying the development of the malignant adrenocortical tumor in a subject (such as preventing metastasis of a tumor), increasing survival (for example, overall survival, relapse-free survival, or metastasis-free survival, such as increased survival time compared to in the absence of treatment), or combinations thereof. Treatment of a tumor also includes reducing signs or symptoms associated with the presence of such a tumor (for example by reducing the size or volume of the tumor or a metastasis thereof). Such reduced growth can in some examples decrease or slow metastasis of the tumor, or reduce the size or volume of the tumor by at least 10%, at least 20%, at least 50%, or at least 75%. Increased survival can include e.g., survival time of at least about 50 months from time of diagnosis, such as about 60 months, about 80 months, about 100 months, about 120 months or about 150 months from time of diagnosis or first treatment.

In some examples, the methods of treatment include selecting a subject in need of treatment, such as a subject that exhibits one or more signs or symptoms known to one of skill in the art to be associated with a malignant adrenocortical tumor, such as one or more signs or symptoms associated with ACC. In some examples, the treatment methods include screening a subject for ACC prior to administering a disclosed treatment. In particular examples, the subject is screened to determine if the adrenocortical tumor is malignant, indicating ACC, or benign. Examples of methods that can be used to screen for ACC include those disclosed herein, including use of a urine sample and/or a combination procedures including evaluating a urine sample (e.g., for the presence of the disclosed metabolites), performing ultrasound, obtaining a tissue biopsy, and/or evaluating serum blood levels for indicators of a malignant adrenocortical tumor. If urine is used, 0.5 to 2 ml of urine is collected and deproteinated. If blood or a fraction thereof (such as serum) is used, 1-100 μl of blood is collected. Serum can either be used directly or fractionated using filter cut-offs to remove high molecular weight proteins. If desired, the serum can be frozen and thawed before use. If a tissue biopsy sample is used, 1-100 μg of tissue is obtained, for example using a fine needle aspirate.

Identification of subjects with the same medical condition, such as a malignant adrenocortical tumor, including ACC, can also be accomplished by selecting all patients with the same diagnosis within electronic health records (EHR). EHRs are simply individual health records in a digitized format that can be accessed via a computer or computer-based system over a network. EHRs are designed to keep information about each encounter with the patient. For example, EHRs may include a person's health characteristics, medical history, past and current diagnoses, lab reports and results, x-rays, photographs, prescribed medication, billing and insurance information, contact information, demographics, and the like.

In some embodiments, the agent is a specific binding agent, such as an antibody, inhibitory nucleic acid (e.g., antisense compound, siRNA, ribozyme) or small molecule inhibitor, that decreases the presence of the target metabolite. Methods of preparing antibodies against a specific target molecule are well known in the art. In some embodiments, an enzyme found to produce the ACC metabolite can be used to produce antibodies which are immunoreactive or specifically bind to an epitope of the enzyme to reduce the levels of the AAC metabolite. Polyclonal antibodies, antibodies which consist essentially of pooled monoclonal antibodies with different epitopic specificities, as well as distinct monoclonal antibody preparations and chimeric antibodies are included. The preparation of polyclonal antibodies is well known to those skilled in the art. See, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992. The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988).

Therapeutic agents are agents that when administered in therapeutically effective amounts induce the desired response (e.g., treatment of ACC). In one example, therapeutic agents are specific binding agents that bind with higher affinity to a molecule of interest, than to other molecules. Examples of specific binding agents include antisense compounds (such as antisense oligonucleotides, siRNAs, miRNAs, shRNAs and ribozymes), antibodies, ligands, recombinant proteins, peptide mimetics, and soluble receptor fragments. Methods of making antisense compounds that can be used clinically are known in the art. In addition, antisense compounds may be commercially available. Exemplary commercially available antisense compounds are available from Santa Cruz Biotechnology, Inc (Santa Cruz, Calif. 95060).

Further examples of specific binding agents include antibodies. Methods of making antibodies that can be used clinically are known in the art and described herein. Such agents can be administered in therapeutically effective amounts to subjects in need thereof, such as a subject having cancer.

### D. Methods for Screening Therapeutic Agents for the Treatment of a Malignant Adrenocortical Tumor

Also provided is the use of the level of at least one metabolite associated with a malignant adrenocortical tumor for screening therapeutic agents for the treatment of a patient with a malignant adrenocortical tumor, including ACC, wherein the at least one metabolite is creatine riboside, Nε,Nε,Nε-trimethyl-L-lysine, L-tryptophan, and/or 3-methylhistidine, and wherein an agent that decreases the level of creatine riboside and/or increases the level of Nε,Nε,Nε-trimethyl-L-lysine, L-tryptophan, and/or 3-methylhistidine is a therapeutic agent for the treatment of a malignant adrenocortical tumor, including ACC. For example, if a metabolite level is elevated in a malignant adrenocortical tumor, including ACC, and an agent that decreases the level by at least 20%, such as at least 30%, at least 40%, at least 50%, at least 60%, or at least 70%, including about a 20%, about a 25, about a 30%, about a 40%, about a 50%, about a 60%, about a 70%, about a 80%, or about a 90% decrease, including a 20% to 30% decrease, a 40% to 50% decrease, a 60% to 80% decrease, or a 80% to 90% decrease, such as a 20%, 30%, 35%, 40%, 45%, 50%, 55%, 60%, 65%, 70%, 75%, 80%, 85%, 90%, or 95% decrease indicates an effective treatment. Further, if a metabolite level is decreased in a malignant adrenocortical tumor, including ACC, and an agent that increases such level by at least 20%, such as at least 30%, at least 40%, at least 50%, at least 60%, or at least 70%, including about a 20%, about a 25, about a 30%, about a 40%, about a 50%, about a 60%, about a 70%, about a 80%, or about a 90% increase, including a 20% to 30% increase, a 40% to 50% increase, a 60% to 80% increase, or a 80% to 90% increase, such as a 20%, 30%, 35%, 40%, 45%, 50%, 55%, 60%, 65%, 70%, 75%, 80%, 85%, 90%, or 95% increase indicates an effective treatment.

In some examples, an effective treatment is an at least 20%, such as at least 30%, at least 40%, at least 50%, at least 60%, or at least 70%, including about a 20%, about a 25, about a 30%, about a 40%, about a 50%, about a 60%, about a 70%, about a 80%, about a 90% decrease, including a 20% to 30% decrease, a 40% to 50% decrease, a 60% to 80% decrease, or a 80% to 90% decrease, such as a 20%, 30%, 35%, 40%, 45%, 50%, 55%, 60%, 65%, 70%, 75%, 80%, 85%, 90%, or 95% decrease in creatine riboside is measured. In some examples, an effective treatment is one in which an at least 20%, such as at least 30%, at least 40%, at least 50%, at least 60%, or at least 70%, including about a 20%, about a 25, about a 30%, about a 40%, about a 50%, about a 60%, about a 70%, about a 80%, about a 90% increase, including a 20% to 30% increase, a 40% to 50% increase, a 60% to 80% increase, or a 80% to 90% increase, such as a 20%, 30%, 35%, 40%, 45%, 50%, 55%, 60%, 65%, 70%, 75%, 80%, 85%, 90%, or 95% increase in Nε,Nε,Nε-trimethyl-L-lysine, L-tryptophan, and/or 3-methylhistidine is measured.

### E. Adrenal Tumors

Adrenal tumors can be benign or malignant adrenal tumors. Malignant adrenal tumors include neuroblastoma, adrenocortical carcinoma (ACC), and a minority of adrenal pheochromocytomas. Most adrenal pheochromocytomas and all adrenocortical adenomas are benign tumors, which do not metastasize or invade nearby tissues, but which may still cause significant health problems by giving rise to hormonal imbalances. Disclosed herein are particular metabolites which can be used to characterize an adrenocortical tumor as well as diagnosis a subject with a malignant adrenal tumor, such as ACC as these molecules are differentially present in malignant adrenal cortex tumors as compared to benign adrenal cortex tumors.

Adrenocortical carcinoma is an aggressive cancer originating in the cortex (steroid hormone-producing tissue) of the adrenal gland. Adrenocortical carcinoma is a rare tumor with an incidence of 1-2 per million population annually. Adenocortical carcinoma is often associated with hormonal syndromes which can occur in patients with steroid hormone-producing (“functional”) tumors, including Cushing's syndrome, Conn syndrome, virilization and feminization. Due to their location deep in the retroperitneum, most adrenocortical carcinomas are not diagnosed until they have grown quite large. They frequently invade large vessels, such as the renal vein and inferior vena cava, as well as metastasizing via the lymphatics and through the blood to the lungs and other organs. The most effective treatment currently is surgery, although this is not feasible for many patients, and the overall prognosis of the disease is poor. Chemotherapy, radiation therapy and hormonal therapy may also be employed in the treatment of this disease.

In contrast, adrenocortical adenomas are benign tumors of the adrenal cortex which are extremely common (present in 1-10% of persons at autopsy). The clinical significance of these neoplasms is twofold. First, they have been detected as incidental findings with increasing frequency in recent years, due to the increasing use of CT scans and magnetic resonance imaging in a variety of medical settings. This can result in expensive additional testing and invasive procedures to rule out the slight possibility of an early adrenocortical carcinoma. Second, a minority (about 15%) of adrenocortical adenomas are “functional”, meaning that they produce glucocorticoids, mineralcorticoids, and/or sex steroids, resulting in endocrine disorders such as Cushing's syndrome, Conn's syndrome (hyperaldosteronism), virilization of females, or feminization of males. Functional adrenocortical adenomas are surgically curable.

Most of the adrenocortical adenomas are less than 2 cm in greatest dimension and less than 50 g in weight. However, size and weight of the adrenal cortical tumors are no longer considered to be a reliable sign of benignity or malignancy. Grossly, adrenocortical adenomas are encapsulated, well-circumscribed, solitary tumors with solid, homogeneous yellow-cut surface. Necrosis and hemorrhage are rare findings.

Pheochromocytoma is a neoplasm composed of cells similar to the chromaffin cells of the mature adrenal medulla. Pheochromocytomas occur in patients of all ages, and may be sporadic, or associated with a hereditary cancer syndrome, such as multiple endocrine neoplasia (MEN) types IIA and IIB, neurofibromatosis type I, or von Hippel-Lindau syndrome. Only 10% of adrenal pheochromocytomas are malignant, while the rest are benign tumors. The most clinically important feature of pheochromocytomas is their tendency to produce large amounts of the catecholamine hormones epinephrine (adrenaline) and norepinephrine. This may lead to potentially life-threatening high blood pressure, or cardiac arrythmias, and numerous symptoms such as headache, palpitations, anxiety attacks, sweating, weight loss and tremor. Diagnosis is often confirmed through urinary measurement of catecholamine metabolites. Typically, pheochromocytomas are initially treated with anti-adrenergic drugs to protect against catecholamine overload, with surgery employed to remove the tumor once the patient is medically stable.

### F. Administration of Agents

Agents can be administered to a subject in need of treatment using any suitable means known in the art. Methods of administration include, but are not limited to, intraductal, intradermal, intramuscular, intraperitoneal, parenteral, intravenous, subcutaneous, intratumoral, vaginal, rectal, intranasal, inhalation, oral or by gene gun. Intranasal administration refers to delivery of the compositions into the nose and nasal passages through one or both of the nares and can comprise delivery by a spraying mechanism or droplet mechanism, or through aerosolization of the molecules. Administration of the compositions by inhalant can be through the nose or mouth via delivery by spraying or droplet mechanisms. Delivery can be directly to any area of the respiratory system via intubation. Parenteral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Administration can be systemic or local.

Agents can be administered in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose required will vary from subject to subject depending on the species, age, weight and general condition of the subject, the particular therapeutic agent being used and its mode of administration. An appropriate dose can be determined by one of ordinary skill in the art using only routine experimentation.

In some embodiments, the therapeutic agent is a nucleic acid-based therapeutic agent. A nucleic acid-based therapeutic agent can be administered to a subject by any suitable route. In some examples, the agents are administered using an enteral or parenteral administration route. Suitable enteral administration routes include, for example, oral, rectal, or intranasal delivery. Suitable parenteral administration routes include, for example, intravascular administration (such as intravenous bolus injection, intravenous infusion, intra-arterial bolus injection, intra-arterial infusion and catheter instillation into the vasculature); subcutaneous injection or deposition, including subcutaneous infusion (such as by osmotic pumps); direct application to the tissue of interest, for example by a catheter or other placement device (e.g., a suppository or an implant comprising a porous, non-porous, or gelatinous material); and inhalation. Particularly suitable administration routes are injection, infusion and direct injection into a target tissue.

In some embodiments, liposomes are used to deliver a compound to a subject. Liposomes can also increase the blood half-life of the gene products or nucleic acids. Suitable liposomes for use in the invention can be formed from standard vesicle-forming lipids, which generally include neutral or negatively charged phospholipids and a sterol, such as cholesterol. The selection of lipids is generally guided by consideration of several factors, such as the desired liposome size and half-life of the liposomes in the blood stream. A variety of methods are known in the art for preparing liposomes (see, for example, Szoka et al., Ann. Rev. Biophys. Bioeng. 9:467, 1980; and U.S. Pat. Nos. 4,235,871; 4,501,728; 4,837,028; and 5,019,369). In some embodiments, polymers can be used to deliver a compound to a subject. Appropriate doses of small molecule agents depend upon a number of factors known to those of ordinary skill in the art, e.g., a physician. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram).

### G. Combination Treatment Methods

The disclosed methods for inhibiting or treating malignant adrenocortical tumors can be used alone or can be accompanied by administration of other anti-cancer agents or therapeutic treatments (such as surgical resection of a tumor or radiation therapy). Any suitable anti-cancer agent can be administered to a patient as part of a treatment regimen that includes inhibiting or treating a malignant adrenocortical tumor. Exemplary anti-cancer agents include, but are not limited to, chemotherapeutic agents, such as, for example, mitotic inhibitors, alkylating agents, anti-metabolites, intercalating antibiotics, growth factor inhibitors, cell cycle inhibitors, enzymes, topoisomerase inhibitors, anti-survival agents, biological response modifiers, anti-hormones (e.g. anti-androgens) and anti-angiogenesis agents. Other anti-cancer treatments include radiation therapy and antibodies that specifically target cancer cells.

Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine).

Examples of antimetabolites include folic acid analogs (such as methotrexate), pyrimidine analogs (such as 5-FU or cytarabine), and purine analogs, such as mercaptopurine or thioguanine.

Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase).

Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide).

Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testerone proprionate and fluoxymesterone).

Examples of many of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, while some more newer drugs include Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol.

In some examples, the chemotherapy regimen includes mitotane (an inhibitor of steroid synthesis which is toxic to cells of the adrenal cortex) as well as standard cytotoxic drugs. For example, an exemplary regimen consists of cisplatin, doxorubicin, etoposide, and mitotane. In some examples, the endocrine cell toxin streptozotocin is included with the chemotherapeutic. In further examples, hormonal therapy with steroid synthesis inhibitors such as aminoglutethimide is used in a palliative manner to reduce the symptoms of hormonal syndromes associated with the ACC.

When used in combination with the administration of one of the disclosed therapeutic agents targeting one or more of the metabolites associated with a malignant adrenocortical tumor (e.g., associated with ACC), the additional treatment methods described above can be administered or performed prior to, at the same time, or following the disclosed anti-tumor therapy as appropriate for the particular patient, the additional symptoms associated with the ACC (e.g., hormonal symptoms, conditions and related diseases) and the specific combination of therapies.

H. Detecting Metabolites Associated with Malignant Adrenocortical Tumors Metabolites associated with a malignant adrenocortical tumor can be detected using a number of methods including those disclosed herein. In some embodiments, metabolite profiles are used to characterize and diagnose malignant adrenocortical tumors and to predict the prognosis and develop potential therapies for patients with malignant adrenocortical tumors, such as to treat ACC. Thus, the disclosed methods can include evaluating metabolites, such as those provided in the Examples below. In some embodiments, the methods provided herein include evaluating levels of creatine riboside, Nε,Nε,Nε-trimethyl-L-lysine, L-tryptophan, and/or 3-methylhistidine in a urine sample obtained from a subject either at risk of acquiring or having an adrenocortical tumor. In some examples, the specific metabolites are quantified. Any one of a number of methods for detecting metabolites can be used, including LCMS, ELISA, SELDI, Western blot, array, chemoluminiscence- or fluorescence-based assays. Exemplary kits and assays are explained in detail below.

### IV. Kits and Assays

Provided by this disclosure are kits that can be used to characterize adrenocortical tumors, such as benign or malignant, and thus, diagnose, prognose, or treat ACC. The disclosed kits can include instructional materials disclosing means of use of the compositions in the kit. The instructional materials can be written, in an electronic form (such as a computer diskette or compact disk) or can be visual (such as video files).

Kits are provided that can be used in the therapies and diagnostic assays disclosed herein. For example, kits can include one or more of the disclosed therapeutic compositions (such as a composition including one or more of the inhibitory nucleic acids or antibodies directed to one or more metabolites upregulated in a subject with a malignant adrenocortical tumor), one or more of the disclosed metabolite ACC profile signatures, or combinations thereof. In one example, the kit further includes one or more chemotherapeutic agents, such as one or more of cisplatin, doxorubicin, etoposide, and mitotane. One skilled in the art will appreciate that the kits can include other agents to facilitate the particular application for which the kit is designed.

In some examples, a kit is provided for detecting one or more of the disclosed metabolites in a biological sample, such as a urine sample. Kits for detecting a malignant adrenocortical tumor, including detecting ACC, can include one or more nucleic acid or antibody probes that specifically bind to the molecules. In an example, a kit includes an array with one or more malignant adrenocortical tumor metabolites and controls, such as positive and negative controls. In other examples, kits include antibodies that specifically bind to one of the disclosed metabolites herein. In some examples, the antibody is labeled (for example, with a fluorescent, radioactive, or an enzymatic label). Such a kit can additionally contain means of detecting a label (such as enzyme substrates for enzymatic labels, filter sets to detect fluorescent labels, appropriate secondary labels such as a secondary antibody, or the like), as well as buffers and other reagents routinely used for the practice of a particular diagnostic method.

In some examples, sensitive assays, such as LCMS-, ELISA-, chemoluminiscence- or fluorescence-based kits, are disclosed which allow differentiation between benign and malignant adrenal neoplasm for screening and diagnosis in a clinical lab set up. Quantitative spectroscopic methods, such as SELDI, can be used to analyze metabolite levels in a sample. In one example, surface-enhanced laser desorption-ionization time-of-flight (SELDI-TOF) mass spectrometry is used to detect metabolites, for example by using the ProteinChip™ (Ciphergen Biosystems, Palo Alto, Calif.). Such methods are known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as malignant adrenocortical tumormetabolites. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as malignant adrenocortical metabolites) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind creatine riboside, Nε,Nε,Nε-trimethyl-L-lysine, L-tryptophan, and 3-methylhistidine. In other examples, the chromatographic surface consists essentially of, or consists of, antibodies that specifically bind creatine riboside, Nε,Nε,Nε-trimethyl-L-lysine, L-tryptophan, and/or 3-methylhistidine. In some examples, the chromatographic surface includes antibodies that bind other molecules, such as control proteins (e.g., creatinine, albumin, prealbumin, and/or transferrin).

In another example, antibodies are immobilized onto the surface using a binding support. The chromatographic surface is incubated with a sample, such as a urine sample from a subject with an adrenocortical tumor. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

In some examples, the methods disclosed herein can be performed in the form of various immunoassay formats, including homogeneous or heterogeneous immunoassays. In homogeneous immunoassays, both the immunological reaction between an antigen and an antibody and the detection are carried out in a homogeneous reaction. Heterogeneous immunoassays include at least one separation step, which allows the differentiation of reaction products from unreacted reagents. A variety of immunoassays can be used to detect one or more of the molecules capable of distinguishing a benign adrenocortical tumor from a malignant adrenocortical tumor. In one example, at least one or more of the following are detected with a disclosed immunoassay: creatine riboside, Nε,Nε,Nε-trimethyl-L-lysine, L-tryptophan, and/or 3-methylhistidine. In some examples, the disclosed immunoassay includes at least one, such as two, three, four or more molecules associated with a malignant adrenocortical tumor, such ACC.

Homogeneous immunoassays include, for example, the Enzyme Multiplied Immunoassay Technique (EMIT), which typically includes a biological sample comprising the molecules to be measured, enzyme-labeled molecules of the biomarkers to be measured, specific antibody or antibodies binding the biomarkers to be measured, and a specific enzyme chromogenic substrate. In a typical EMIT, excess of specific antibodies is added to a biological sample. If the biological sample contains the molecules to be detected, such molecules bind to the antibodies. A measured amount of the corresponding enzyme-labeled molecules is then added to the mixture. Antibody binding sites not occupied by molecules of the protein in the sample are occupied with molecules of the added enzyme-labeled protein. As a result, enzyme activity is reduced because only free enzyme-labeled protein can act on the substrate. The amount of substrate converted from a colorless to a colored form determines the amount of free enzyme left in the mixture. A high concentration of the protein to be detected in the sample causes higher absorbance readings. Less protein in the sample results in less enzyme activity and consequently lower absorbance readings. Inactivation of the enzyme label when the antigen-enzyme complex is antibody-bound makes the EMIT a useful system, enabling the test to be performed without a separation of bound from unbound compounds as is necessary with other immunoassay methods. A homogenous immunoassay, such as an EMIT, can be used to detect any of the molecules associated with a malignant adrenocortical tumor, such as creatine riboside, Nε,Nε,Nε-trimethyl-L-lysine, L-tryptophan, and/or 3-methylhistidine.

ELISA is a heterogeneous immunoassay and can be used in the methods disclosed herein. The assay can be used to detect protein antigens in various formats. In the “sandwich” format the antigen being assayed is held between two different antibodies. In this method, a solid surface is first coated with a solid phase antibody. The test sample, containing the antigen (e.g., a diagnostic protein), or a composition containing the antigen, such as a urine sample from a subject of interest, is then added and the antigen is allowed to react with the bound antibody. Any unbound antigen is washed away. A known amount of enzyme-labeled antibody is then allowed to react with the bound antigen. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the antigen present in the sample tested.

ELISA can also be used as a competitive assay. In the competitive assay format, the test specimen containing the antigen to be determined is mixed with a precise amount of enzyme-labeled antigen and both compete for binding to an anti-antigen antibody attached to a solid surface. Excess free enzyme-labeled antigen is washed off before the substrate for the enzyme is added. The amount of color intensity resulting from the enzyme-substrate interaction is a measure of the amount of antigen in the sample tested. A heterogeneous immunoassay, such as an ELISA, can be used to detect any molecules associated with a benign or malignant adrenocortical tumor, such ACC.

Immunoassay kits are also disclosed herein. These kits include, in separate containers (a) monoclonal antibodies having binding specificity for the metabolites used in the characterization/diagnosis of an adrenocortical tumor, such as a malignant adrenocortical tumor (e.e., ACC); and (b) and anti-antibody immunoglobulins. This immunoassay kit may be utilized for the practice of the various methods provided herein. The monoclonal antibodies and the anti-antibody immunoglobulins can be provided in an amount of about 0.001 mg to 100 grams, and more preferably about 0.01 mg to 1 gram. The anti-antibody immunoglobulin may also be a polyclonal immunoglobulin, protein A or protein G or functional fragments thereof, which may be labeled prior to use by methods known in the art. In several embodiments, the immunoassay kit includes one, two, three or four or more antibodies that specifically bind to molecules associated with a malignant adrenocortical tumor, such as creatine riboside, Nε,Nε,Nε-trimethyl-L-lysine, L-tryptophan, and/or 3-methylhistidine. The immunoassay kit can also include one or more antibodies that specifically bind to one or more of these molecules. Thus, the kits can be used to detect one or more different molecules associated a malignant adrenocortical tumor and thus, ACC. In one specific example, a kit includes four antibodies, an antibody to creatine riboside, an antibody to Nε,Nε,Nε-trimethyl-L-lysine, an antibody to L-tryptophan, and an antibody to 3-methylhistidine.

Immunoassays for polysaccharides and proteins differ in that a single antibody is used for both the capture and indicator roles for polysaccharides due to the presence of repeating epitopes. In contrast, two antibodies specific for distinct epitopes are required for immunoassay of proteins. Exemplary samples include biological samples obtained from subjects including, but not limited to, urine samples.

In one particular example, a quantitative ELISA is constructed for detection of at least one of (such as all four of) creatine riboside, Nε,Nε,Nε-trimethyl-L-lysine, L-tryptophan, and/or 3-methylhistidine. These immunoassays utilize antibodies, such as mAbs commercially available. Since a polysaccharide is a polyvalent repeating structure, a single mAb may be used for both the capture and indicator phases of an immunoassay. The only requirement is that the mAb have a sufficient affinity. A mAb with an affinity of about 0.5 μM has sufficient affinity.

### V. Capture Device Methods

The disclosed methods can be carried out using a sample capture device, such as a lateral flow device (for example a lateral flow test strip) that allows detection of one or more molecules, such as those described herein.

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept)avidin. The assays are often performed with test strips in which a specific binding pair member is attached to a mobilizable material (such as a metal sol or beads made of latex or glass) or an immobile substrate (such as glass fibers, cellulose strips or nitrocellulose membranes). Particular examples of some of these assays are shown in U.S. Pat. Nos. 4,703,017; 4,743,560; and U.S. Pat. No. 5,073,484 (incorporated herein by reference). The test strips include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853; WO 88/08534; and EP-A 0 299 428 (incorporated herein by reference). There are a number of commercially available lateral-flow type tests and patents disclosing methods for the detection of large analytes (MW greater than 1,000 Daltons) as the analyte flows through multiple zones on a test strip. Examples are found in U.S. Pat. No. 5,229,073 (measuring plasma lipoprotein levels), and U.S. Pat. Nos. 5,591,645; 4,168,146; 4,366,241; 4,855,240; 4,861,711; 5,120,643; European Patent No. 0296724; WO 97/06439; WO 98/36278; and WO 08/030546 (each of which are herein incorporated by reference). Multiple zone lateral flow test strips are disclosed in U.S. Pat. No. 5,451,504, U.S. Pat. No. 5,451,507, and U.S. Pat. No. 5,798,273 (incorporated by reference herein). U.S. Pat. No. 6,656,744 (incorporated by reference) discloses a lateral flow test strip in which a label binds to an antibody through a streptavidin-biotin interaction.

In particular examples, the methods disclosed herein include application of a biological sample (such as urine) from a test subject to a lateral flow test device for the detection of one or more molecules (such as one or more molecules associated with a malignant adrenocortical tumor, such as ACC, for example, combinations of molecules as described above) in the sample. The lateral flow test device includes one or more antibodies (such as antibodies that bind one or more of the molecules associated with a malignant adrenocortical tumor (such as ACC) and not a benign adrenocortical tumor) at an addressable location. In a particular example, the lateral flow test device includes antibodies that bind at least one of creatine riboside, Nε,Nε,Nε-trimethyl-L-lysine, L-tryptophan, and/or 3-methylhistidine. The addressable locations can be, for example, a linear array or other geometric pattern that provides diagnostic information to the user. The binding of one or more molecules in the sample to the antibodies present in the test device is detected and the presence or amount of one or more molecules in the sample of the test subject is compared to a control, wherein a change in the presence or amount of one or more molecules in the sample from the test subject as compared to the control/reference value indicates that the subject has a malignant adrenocortical tumor, such as ACC. In one particular example, an increase in creatine riboside and a decrease in Nε,Nε,Nε-trimethyl-L-lysine, L-tryptophan, and 3-methylhistidine, such as at least a 2-fold increase in creatine riboside and an at least 1.8 fold decrease in Nε,Nε,Nε-trimethyl-L-lysine, L-tryptophan, and 3-methylhistidine compared to the control/reference value indicates that the subject has a malignant adrenocortical tumor, such as ACC.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more molecules disclosed herein. Thus these zones can be viewed as functional sectors or functional regions within the test device.

In general, a fluid sample is introduced to the strip at the proximal end of the strip, for instance by dipping or spotting. A sample is collected or obtained using methods well known to those skilled in the art. The sample containing the particular molecules to be detected may be obtained from a urine sample. In other examples, a blood or salvia sample is used. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

Another common feature to be considered in the use of assay devices is a means to detect the formation of a complex between an analyte (such as one or more molecules described herein) and a capture reagent (such as one or more antibodies). A detector (also referred to as detector reagent) serves this purpose. A detector may be integrated into an assay device (for example included in a conjugate pad, as described below), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest, for example those described herein).

In other instances, a detector reagent collectively includes an unlabeled first binding partner specific for the analyte and a labeled second binding partner specific for the first binding partner and so forth. Thus, the detector can be a labeled antibody specific for a metabolite described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab(Fc)) that is present in a secondary capture area.

### Flow-Through Device Construction and Design

Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030546. A flow-through device involves a capture reagent (such as one or more antibodies) immobilized on a solid support, typically, a membrane (such as, nitrocellulose, nylon, or PVDF). Characteristics of useful membranes have been previously described; however, it is useful to note that in a flow-through assay capillary rise is not a particularly important feature of a membrane as the sample moves vertically through the membrane rather than across it as in a lateral flow assay. In a simple representative format, the membrane of a flow-through device is placed in functional or physical contact with an absorbent layer (see, e.g., description of “absorbent pad” below), which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample (such as a bodily fluid sample) is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more molecules described herein) can specifically bind to the immobilized capture reagent (such as one or more antibodies). Where detection of an analyte-capture reagent complex is desired, a detector reagent (such as labeled antibodies that specifically bind one or more molecules) can be added with the sample or a solution containing a detector reagent can be added subsequent to application of the sample. If an analyte is specifically bound by capture reagent, a visual representative attributable to the particular detector reagent can be observed on the surface of the membrane. Optional wash steps can be added at any time in the process, for instance, following application of the sample, and/or following application of a detector reagent.

### Lateral Flow Device Construction and Design

Lateral flow devices are disclosed herein. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,168,146; 4,313,734; 4,366,241; 4,435,504; 4,775,636; 4,703,017; 4,740,468; 4,806,311; 4,806,312; 4,861,711; 4,855,240; 4,857,453; 4,861,711; 4,943,522; 4,945,042; 4,496,654; 5,001,049; 5,075,078; 5,126,241; 5,120,643; 5,451,504; 5,424,193; 5,712,172; 6,555,390; 6,258,548; 6,699,722; 6,368,876 and 7,517,699; EP 0810436; EP 0296724; WO 92/12428; WO 94/01775; WO 95/16207; WO 97/06439; WO 98/36278; and WO 08/030546, each of which is incorporated by reference. Further, there are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte.

Many lateral flow devices are one-step lateral flow assays in which a biological fluid is placed in a sample area on a bibulous strip (though non-bibulous materials can be used, and rendered bibulous, e.g., by applying a surfactant to the material), and allowed to migrate along the strip until the liquid comes into contact with a specific binding partner (such as an antibody) that interacts with an analyte (such as one or more molecules) in the liquid. Once the analyte interacts with the binding partner, a signal (such as a fluorescent or otherwise visible dye) indicates that the interaction has occurred. Multiple discrete binding partners (such as antibodies) can be placed on the strip (for example in parallel lines) to detect multiple analytes (such as two or more molecules) in the liquid. The test strips can also incorporate control indicators, which provide a signal that the test has adequately been performed, even if a positive signal indicating the presence (or absence) of an analyte is not seen on the strip.

An exemplary construction and design of lateral flow device is described in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Lateral flow devices have a wide variety of physical formats. Any physical format that supports and/or houses the basic components of a lateral flow device in the proper function relationship is contemplated by this disclosure.

In some embodiments, the lateral flow strip is divided into a proximal sample application pad, an intermediate test result zone, and a distal absorbent pad. The flow strip is interrupted by a conjugate pad that contains labeled conjugate (such as gold- or latex-conjugated antibody specific for the target analyte or an analyte analog). A flow path along strip passes from proximal pad, through conjugate pad, into test result zone, for eventual collection in absorbent pad. Selective binding agents are positioned on a proximal test line in the test result membrane. A control line is provided in test result zone, slightly distal to the test line. For example, in a competitive assay, the binding agent in the test line specifically binds the target analyte, while the control line less specifically binds the target analyte.

In operation of the particular embodiment of a lateral flow device, a fluid sample containing an analyte of interest, such as one or more metabolites described herein (for example, a metabolite listed in Table 3), is applied to the sample pad. In some examples, the sample may be applied to the sample pad by dipping the end of the device containing the sample pad into the sample (such as urine) or by applying the sample directly onto the sample pad.

From the sample pad, the sample passes, for instance by capillary action, to the conjugate pad. In the conjugate pad, the analyte of interest, such as a protein of interest, may bind (or be bound by) a mobilized or mobilizable detector reagent, such as an antibody (such as antibody that recognizes one or more of the molecules described herein). For example, a protein analyte may bind to a labeled (e.g., gold-conjugated or colored latex particle-conjugated) antibody contained in the conjugate pad. The analyte complexed with the detector reagent may subsequently flow to the test result zone where the complex may further interact with an analyte-specific binding partner (such as an antibody that binds a particular molecule, an anti-hapten antibody, or streptavidin), which is immobilized at the proximal test line. In some examples, a molecule complexed with a detector reagent (such as gold-conjugated antibody) may further bind to unlabeled, oxidized antibodies immobilized at the proximal test line. The formation of a complex, which results from the accumulation of the label (e.g., gold or colored latex) in the localized region of the proximal test line is detected. The control line may contain an immobilized, detector-reagent-specific binding partner, which can bind the detector reagent in the presence or absence of the analyte. Such binding at the control line indicates proper performance of the test, even in the absence of the analyte of interest. The test results may be visualized directly, or may be measured using a reader (such as a scanner). The reader device may detect color or fluorescence from the readout area (for example, the test line and/or control line).

In another embodiment of a lateral flow device, there may be a second (or third, fourth, or more) test line located parallel or perpendicular (or in any other spatial relationship) to test line in test result zone. The operation of this particular embodiment is similar to that described in the immediately preceding paragraph with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

1. Sample Pad

The sample pad is a component of a lateral flow device that initially receives the sample, and may serve to remove particulates from the sample. Among the various materials that may be used to construct a sample pad (such as glass fiber, woven fibers, screen, non-woven fibers, cellosic fibers or paper), a cellulose sample pad may be beneficial if a large bed volume (e.g., 250 μl/cm²) is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

2. Membrane and Application Solution:

The types of membranes useful in a lateral flow device (such as nitrocellulose (including pure nitrocellulose and modified nitrocellulose), nitrocellulose direct cast on polyester support, polyvinylidene fluoride, or nylon), and considerations for applying a capture reagent to such membranes have been discussed previously.

In some embodiments, membranes comprising nitrocellulose are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

3. Conjugate Pad

The conjugate pad serves to, among other things, hold a detector reagent. Suitable materials for the conjugate pad include glass fiber, polyester, paper, or surface modified polypropylene. In some embodiments, a detector reagent may be applied externally, for example, from a developer bottle, in which case a lateral flow device need not contain a conjugate pad (see, for example, U.S. Pat. No. 4,740,468).

Detector reagent(s) contained in a conjugate pad is typically released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and β-lactose. A mixture of two or more release agents may be used in any given application. In a particular disclosed embodiment, the detector reagent in conjugate pad is a gold-conjugated antibody.

4. Absorbent Pad

The use of an absorbent pad in a lateral flow device is optional. The absorbent pad acts to increase the total volume of sample that enters the device. This increased volume can be useful, for example, to wash away unbound analyte from the membrane. Any of a variety of materials is useful to prepare an absorbent pad, for example, cellulosic filters or paper. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

The following examples are provided to illustrate certain particular features and/or embodiments. These examples should not be construed to limit the disclosure to the particular features or embodiments described.

### EXAMPLES

### Example 1—Materials and Methods

### Urine Samples

Fasting urine samples from patients undergoing adrenal surgery were obtained the morning of surgery and stored at −80° C. Urine samples from patients undergoing experimental treatment for ACC were obtained the morning of drug treatment and stored at −80° C. Demographic, clinical, and pathologic information and samples were collected under an Institutional Review Board (IRB) approved protocol. Clinical characteristics of the study cohort are summarized in Table 1 below. Tumors were classified as ACC if the Weiss criteria was >3 at the initial surgery and/or they developed distant metastasis at follow up. Tumors were classified as benign if the Weiss score was <3 and there was no recurrent or metastatic disease that developed during follow up.

### Global Urinary Metabolomic Analysis and Biomarker Identification.

Urine samples were deproteinated (dilution of 1:5) using a solution of isopropanol/acetonitrile/water (65/30/5) containing 10 μM chloropropamide or α-aminopimelic acid as internal standards for reverse-phase (RP) or hydrophilic interaction liquid chromatography (HILIC), respectively. Supernatants were transferred into 96-well sample plates. All pipetting and dilution were performed using a MICROLAB STARLET automated liquid handler (Hamilton Robotics, Reno, Nev.). For HILIC analysis, 5 μL aliquot of samples were injected in a randomized fashion into a 2.1×50 mm Acquity UPLC BEH amide column (1.7 μm) connected to a Waters XEVO G2 ESI-QTOF mass spectrometer (Waters Corporation, Milford, Mass.). Chromatographic separation was achieved by using a mixture of (A) 10 mM ammonium acetate in 90% acetonitrile (pH=9.0) and (B) 10 mM ammonium acetate in 10% acetonitrile (pH=9.0) as mobile phase. The gradient elution was performed over 10 mM using: 1-60% B in 4 mM, 60-80% B at 8 mM, holding at 80% B to 8.5 mM, returning to initial conditions for column equilibration. Flow rate was maintained at 0.4 ml/min and total run time for each sample was 12.5 min. Column temperature was maintained at 40° C. For RP analysis, samples were further diluted with an equal volume of water before a 5 |μL aliquot of samples was injected into a 2.1×50 mm Acquity UPLC BEH C18 column (1.7 |m). Chromatographic separation was achieved by using a mixture of (A) water containing 1% formic acid and (B) acetonitrile containing 1% formic acid as mobile phase. The gradient elution was performed over 6 mM at a flow rate of 0.4 ml/min using: 1-99% B in 4 mM, holding at 99% B up to 5.0 mM, returning to initial conditions for column equilibration (total run time of 10 mM.). Column temperature was maintained at 40° C. The column was re-equilibrated with 98% A at the end of each run prior to injection of the next sample. Mass spectrometric analysis (for both RP and HILIC chromatography) was performed in both positive and negative ionization modes. Sulfadimethoxine was used as the lock mass (m/z 311.0814+) for accurate mass calibration in real time. MassLynx software (Waters Corporation, Milford, Mass.) was used to acquire mass chromatograms and mass spectral data in centroid format. Chromatographic separation (for both RP and HILIC conditions) was performed on a Waters Acquity H-class UPLC system consisting of a quaternary solvent manager, FTN-solvent manager, and a column manger, all controlled by MassLynx software.

### Targeted Urinary Metabolite Quantitation

Metabolites in the deproteinated urine samples were quantified in multiple reactions monitoring mode on a Waters XEVO TQ-S triple quadrupole mass spectrometer (Water Corporation, Milford, Mass.). α-aminopimelic acid (0.5 μM) was used as internal standard. The following metabolites were quantified by monitoring characteristic fragmentation reactions (in bracket); α-aminopimelic acid (176-112, ESI+), 3-methylhistidine (170→96, ESI+), Nε,Nε,Nε-trimethyl-L-lysine(189→84, ESI+), L-tryptophan (205→118, ESI−), creatine (132→90, ESI+), and creatine riboside (264-132, ESI+). Creatine was used to standardize creatine riboside due to lack of purified standard.

Chromatographic separation was achieved on a 2.1×50 mm Acquity UPLC BEH amide column using the mobile phase as mentioned above. All data were processed using Waters TargetLynx software. Internal standard-normalized area under the peak (response) from serially diluted authentic standard solution was used to build calibration curve for each metabolite. The concentration of metabolite was determined from the calibration curve and divided by creatinine concentration to determine creatinine-normalized excretion of the urine metabolite.

### Quality Control and Normalization

The order of sample injection was randomized to avoid order artifact using the Rand function in Microsoft Excel. Quality control involved running a standard mix prior to samples to monitor instrument performance over time. Internal standards (α-aminopimelic acid, chlorpropamide) were used for HILIC and reverse phase chromatography, respectively. A set of pooled samples was injected at regular intervals during the analysis. The data was assessed with unsupervised principal component analyses. Creatinine concentration was determined by the Jaffe method. In brief, creatinine was assayed colorimetrically after reaction of creatinine with picrate to generate a chromophore in alkaline solution. The absorbance of the creatinine-picrate chromophore was measured at 500 nm in a 96 well microplate. Statistical analyses Samples were classified as ACC or benign adrenal tumor. Data acquired was aligned and deconvoluted by Progenesis software (Durham, N.C. USA). The data was normalized to externally measured creatinine by the Jaffe method (see above). Data was exported and analyzed using a minimal detection rate to eliminate spurious human and instrument variation.

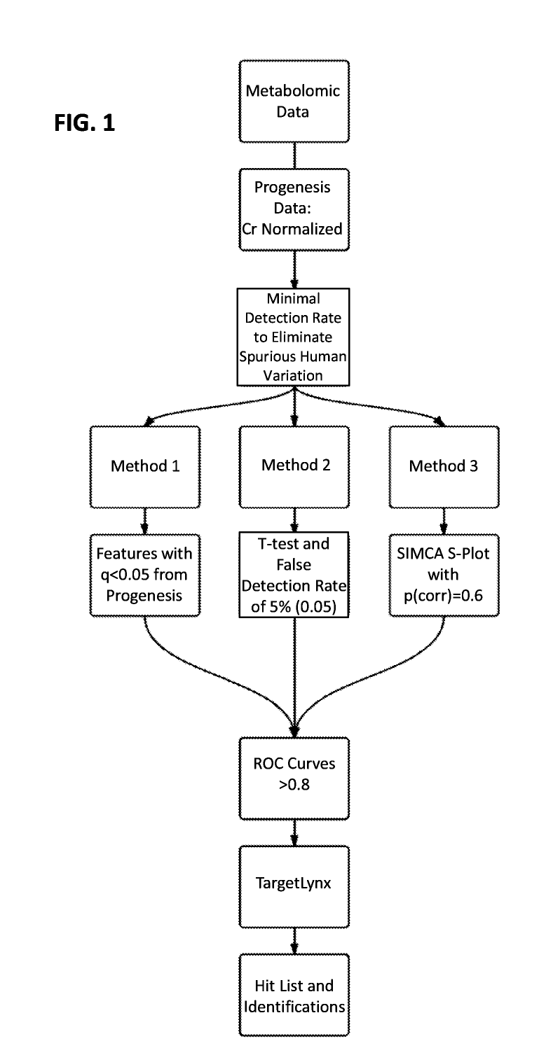

Minimal detection rate was defined as a feature that must be present in at least 40% of samples in each group. The data was analyzed in three separate and complementary methods (FIG. 1). Method 1 used the Progenesis software to compare the groups and a one way ANOVA with a false discovery rate (FDR) of q<0.05. Method 2 utilized a two-tailed T-test and Benjamini-Hochberg test for FDR 5%. Method 3 used SIMCA with the construction of principal component analysis (PCA) and orthogonal partial least squares discriminant analysis (OPLS-DA) plots with a p correlation of 0.5 for intergroup discrimination. Potential features that were statistically significant by any of the three methods were then analyzed using ROCCET software (available on the World Wide Web at domain name roccet.ca) with an AUC cutoff of >0.8 for each feature. Each potential feature was then verified by using TargetLynx software (Waters Corporation, Milford, Mass.). In order to eliminate features related to mitotane administration, PCA and OPLS-DA plots were constructed using SIMCA software. S-plots that did not show a correlation to mitotane ingestion were considered as related to cancer and were included in further analysis. Features were compared by the Mann-Whitney U test. A p-value of less than 0.05 was considered significant.

### Example 2

### Urinary Metabolomics Features of Adrenal Neoplasms

This example demonstrates urinary metabolic features are associated with ACC. Furthermore, four metabolites, creatine riboside, Nε,Nε,Nε-trimethyl-L-lysine, 3-methylhistidine, and tryptophan, are identified as diagnostic small molecule biomarkers for adrenal neoplasms.

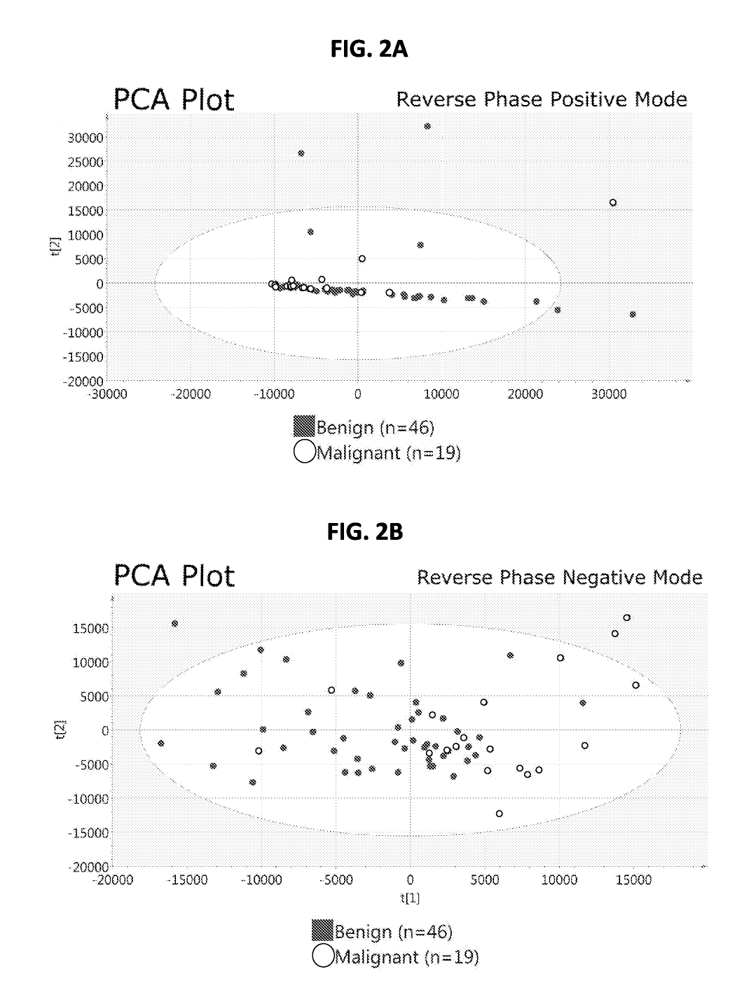

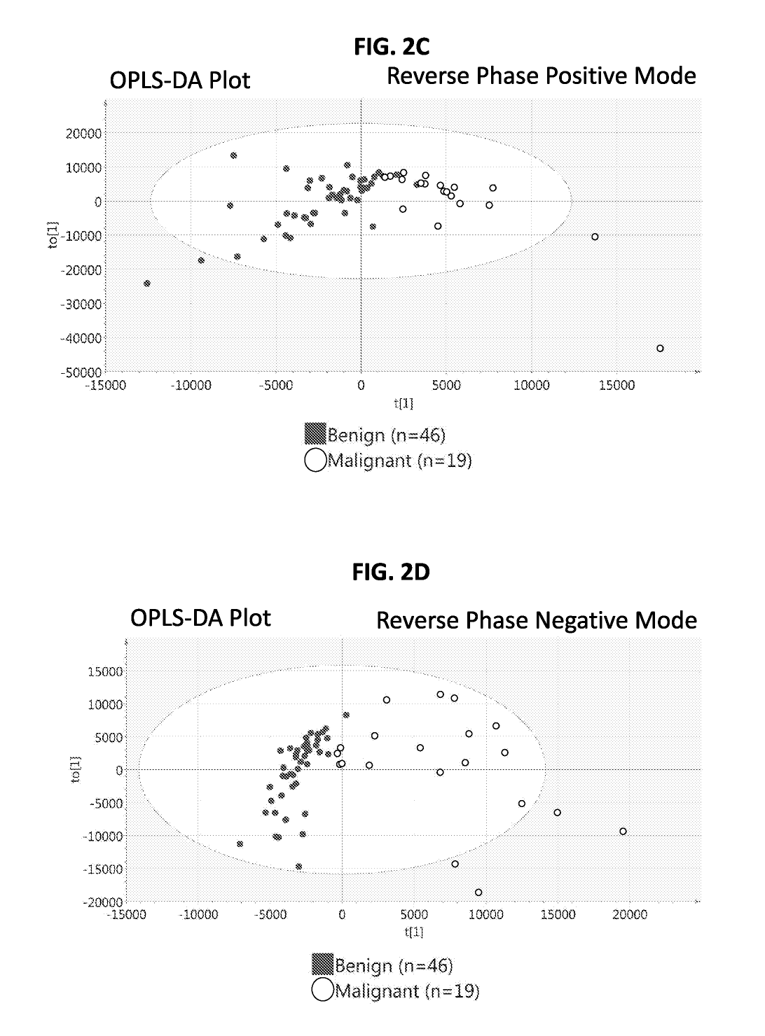

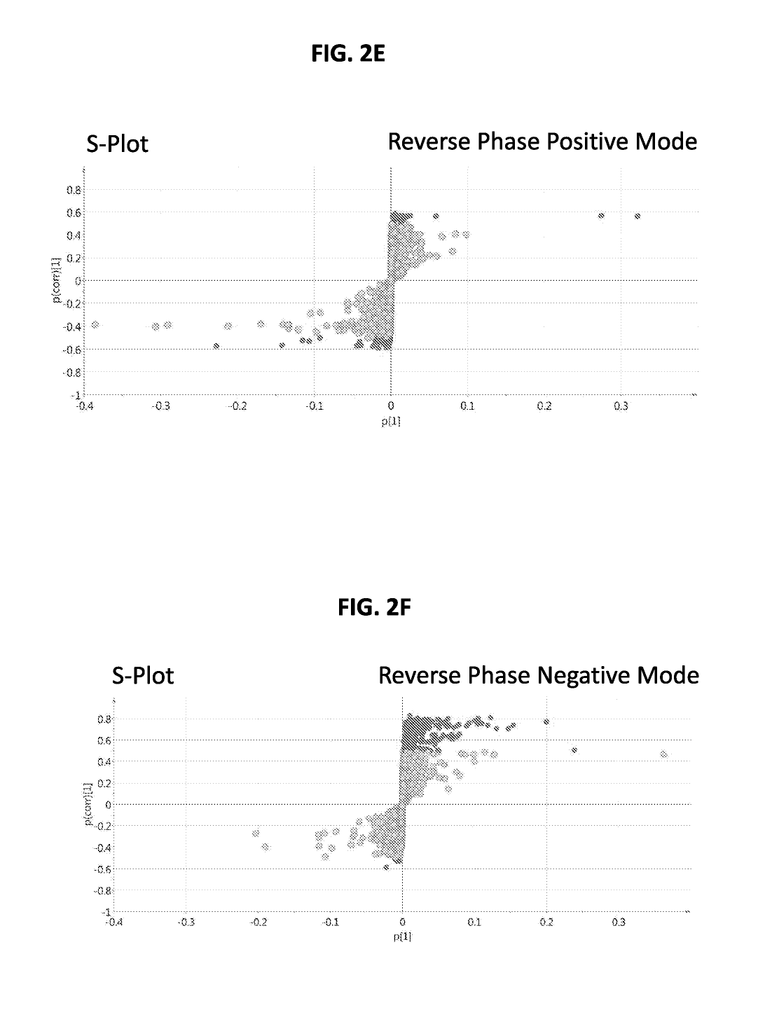

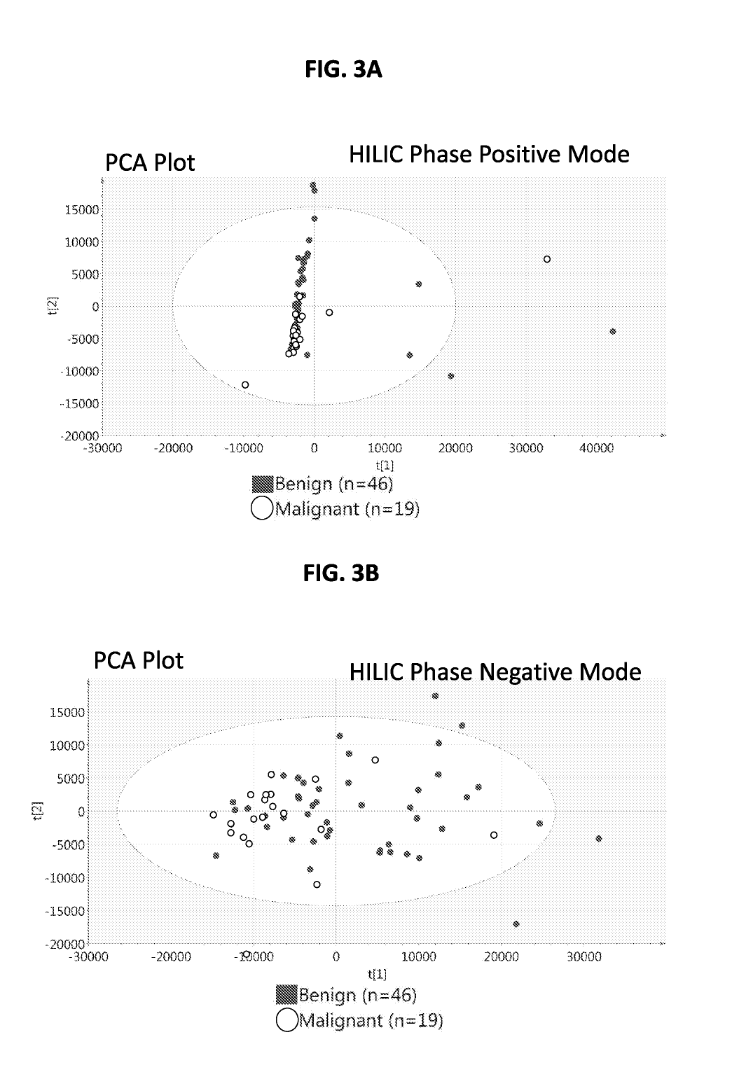

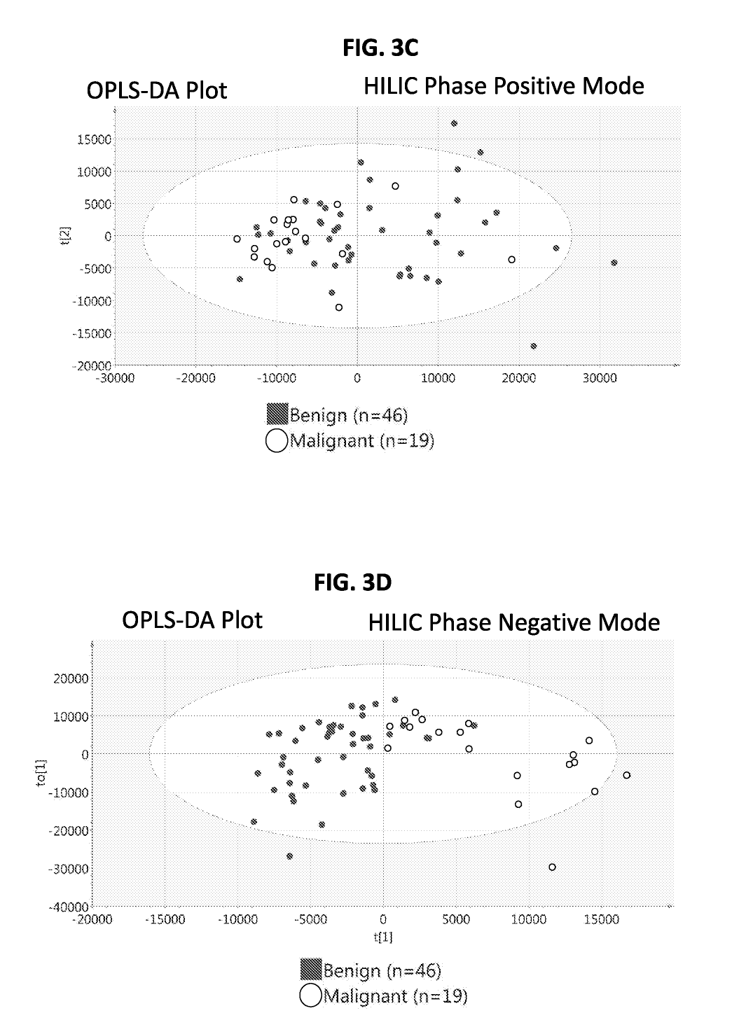

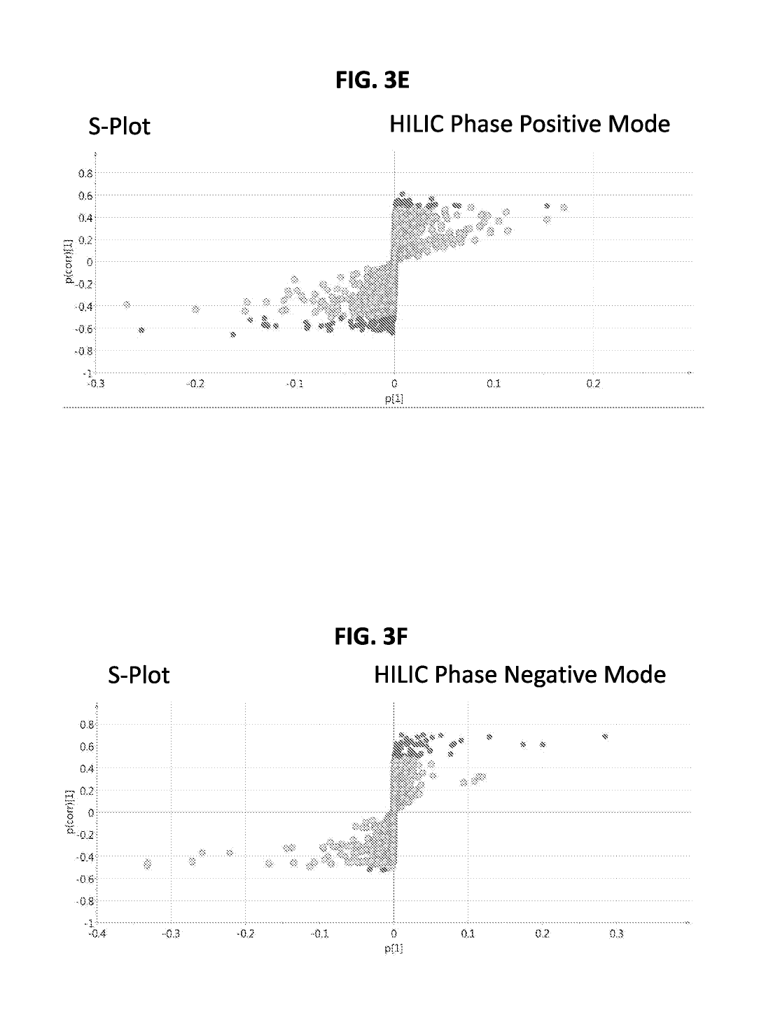

The clinical data are summarized in Tables 1A-1C below. Prepared urine samples were tested and analyzed with multiple methods to reduce false-positive and false-negative results (FIG. 1). Unsupervised PCA plots show discrimination and clustering between patients with ACC and benign adrenal tumors in reverse phase positive, reverse phase negative, HILIC positive, and HILIC negative modes (FIGS. 2A, 2B, 3A, and 3B). Supervised OPLS-DA analysis showed separation and features correlated with ACC and benign adrenal tumors (FIGS. 2C, 2D, 3C, and 3D). S-plot analysis showed features associated with ACC and benign adrenal disease for each mode, respectively (FIGS. 2E, 2F, 3E, and 3F). These data show that patients with ACC and benign adrenal tumors have significantly different urinary metabolites.

TABLE 1A
Clinical Characteristics of Study Cohorts

| | Training | | Independent | |
|---|---|---|---|---|
| | Set | | Set | |
| No. of patients | 19 | 46 | 12 | 45 |
| Age (average ± SD) | 55.68 ± | 47.85 ± | 47.33 ± | 41.94 ± |
| | 10.78 | 15.99 | 15.65 | 17.54 |
| Gender | 11/8 | 26/20 | 6/6 | 30/15 |
| (female/male) | | | | |
| Syndrome: | | | | |
| Cushings | 7 | 24 | 4 | 21 |
| Subclinical | 0 | 0 | 0 | 3 |
| Cushing's | | | | |
| Conn's | 1 | 20 | 1 | 13 |
| Hyperandrogenism | 0 | 0 | 0 | 2 |
| Nonfunctioning | 11 | 2 | 7 | 6 |

TABLE 1B
Pathological and Clinical Characteristics of Patients with Adrenocortical Carcinoma in the Training Set

| Patient | Age at | Age at | | Operation | | Ki67 | Weiss | Distant | |
|---|---|---|---|---|---|---|---|---|---|
| ID | Diagnosis | Operation | Sex | #^{a} | TNM^{b} | (%) | Score | Mets^{c} | Chemotherapy^{d} |
| 1 | 59 | 69 | F | 2 | — | >20% | — | Yes | EDP + mitotane |
| 2 | 44 | 48 | F | 2 | — | >20% | — | Yes | EDP + mitotane |
| 3 | 59 | 65 | M | 2 | — | >20% | — | Yes | EDP + mitotane |
| 4 | 39 | 55 | F | 2 | — | —^{e} | — | Yes | tariquidar, doxorubicin, |
| | | | | | | | | | vincristine, etoposide + |
| | | | | | | | | | mitotane |
| 5 | 49 | 53 | M | 2 | — | >20% | — | Yes | streptozocin + mitotane |
| 6 | 32 | 35 | F | 2 | — | >20% | — | Yes | none |
| 7 | 49 | 52 | F | 1 | IV | <5% | 5/9 | Yes | cisplatin |
| 8 | 52 | 55 | F | 2 | — | >20% | — | Yes | IMC-A12 + mitotane |
| 9 | 49 | 51 | M | 2 | — | —^{e} | — | No | none |
| 10 | 68 | 73 | F | 3 | — | >20% | — | No | mitotane |
| 11 | 34 | 38 | M | 2 | — | >20% | — | Yes | mitotane |
| 12 | 63 | 70 | F | —^{f} | — | >20% | — | Yes | tariquidar, doxorubicin, |
| | | | | | | | | | vincristine, etoposide + |
| | | | | | | | | | mitotane |
| 13 | 58 | 62 | F | —^{f} | — | >20% | — | Yes | OSI-906, EDP, streptozocin + |
| | | | | | | | | | mitotane |
| 14 | 57 | 62 | F | 3 | — | >20% | — | No | mitotane |
| 15 | 55 | 57 | M | 2 | — | >20% | — | Yes | EDP + mitotane |
| 16 | 52 | 57 | F | 2 | — | >20% | — | Yes | mitotane |
| 17 | 39 | 41 | M | 1 | IV | —^{e} | —^{e} | Yes | EDP, cetuximab + mitotane |
| 18 | 50 | 54 | M | 1 | IV | <5% | 5/9 | Yes | AT-101 + mitotane |
| 19 | 63 | 64 | M | 2 | — | >20% | — | Yes | mitotane |

^{a}Number of abdominal operations.
^{b}TNM staging at initial operation (if the initial operation was performed at the NIH).
^{c}Distant metastatic disease at presentation to the NIH and time of the operation.
^{d}Chemotherapy prior to operation (including both neoadjuvant and adjuvant)
^{e}Information not available.
^{f}Patient underwent pulmonary metastasectomy.

TABLE 1C
Pathological and Clinical Characteristics of Patients with Adrenocortical Carcinoma in the Validation Set

| | | Age at | | | | | | | |
|---|---|---|---|---|---|---|---|---|---|
| Patient | Age at | Enrollment/ | | Operation | | Ki67 | Weiss | Distant | |
| ID | Diagnosis | Operation | Sex | #^{a} | TNM^{b} | (%) | Score | Mets^{c} | Chemotherapy^{d} |
| 1 | 50 | 54 | M | 1^{e} | — | — | — | Yes | EDP + mitotane |
| 2 | 64 | 65 | F | —^{e} | — | — | — | Yes | EDP + mitotane |
| 3 | 28 | 31 | F | 1^{e} | — | — | — | Yes | EDP + mitotane, |
| | | | | | | | | | zoledronic acid |
| 4 | 34 | 36 | M | 2^{e} | — | — | — | Yes | EDP + mitotane |
| 5 | 33 | 39 | M | 1^{e} | — | — | — | Yes | EDP + mitotane |
| 6 | 52 | 59 | F | 4^{e} | — | — | — | Yes | mitotane |
| 7 | 43 | 46 | F | 2^{e} | — | — | — | Yes | EDP + mitotane |
| 8 | 15 | 18 | F | 1^{ef} | — | — | — | Yes | Bortezomib, |
| | | | | | | | | | carfilzomib, cisplatin, |
| | | | | | | | | | abraxane |
| 9 | 63 | 66 | M | 2 | — | —^{g} | — | Yes | mitotane |
| 10 | 53 | 53 | M | 1 | IV | —^{h} | —^{h} | Yes | none |
| 11 | 34 | 34 | F | 1 | II | 10% | 5/9 | No | none |

^{a}Number of abdominal operations.
^{b}TNM staging at time of presentation (if the initial operation was performed at the NIH).
^{c}Distant metastatic disease at presentation to the NIH and time of the operation.
^{d}Chemotherapy prior to experimental treatment or operation (including both neoadjuvant and adjuvant)
^{e}Patient was not operable at the time of enrollment and collection of urine specimen.
^{f}Patient underwent resection and radiofrequency ablation of pulmonary metastases.
^{g}Information not available.
^{h}Tumor was not resectable and was left in situ.

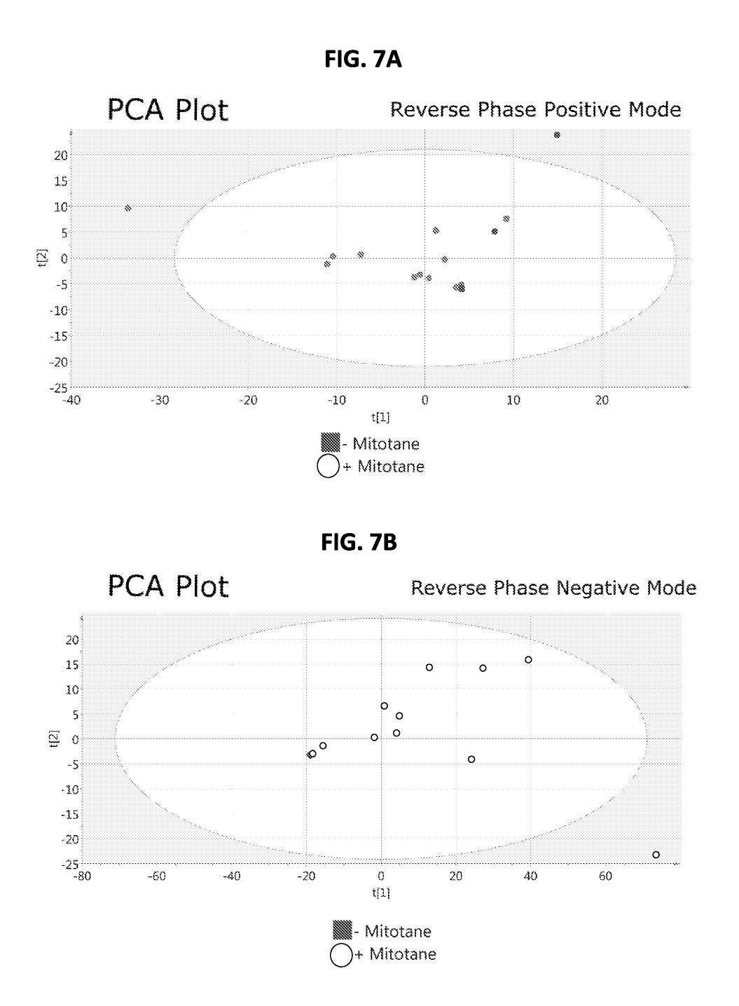

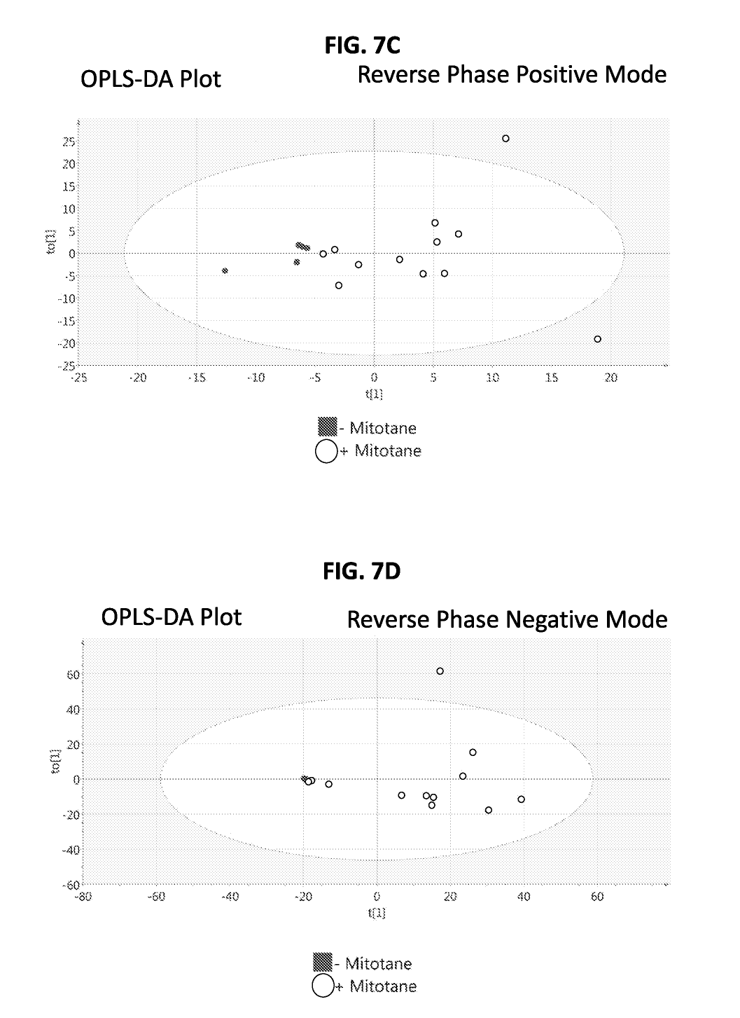

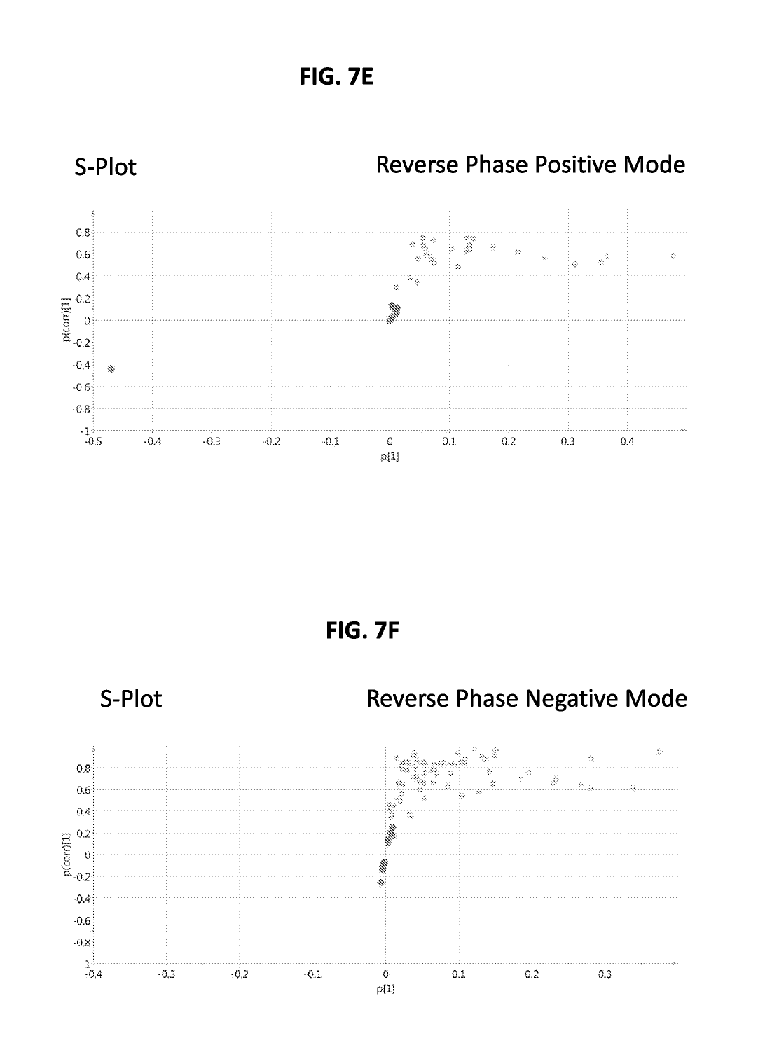

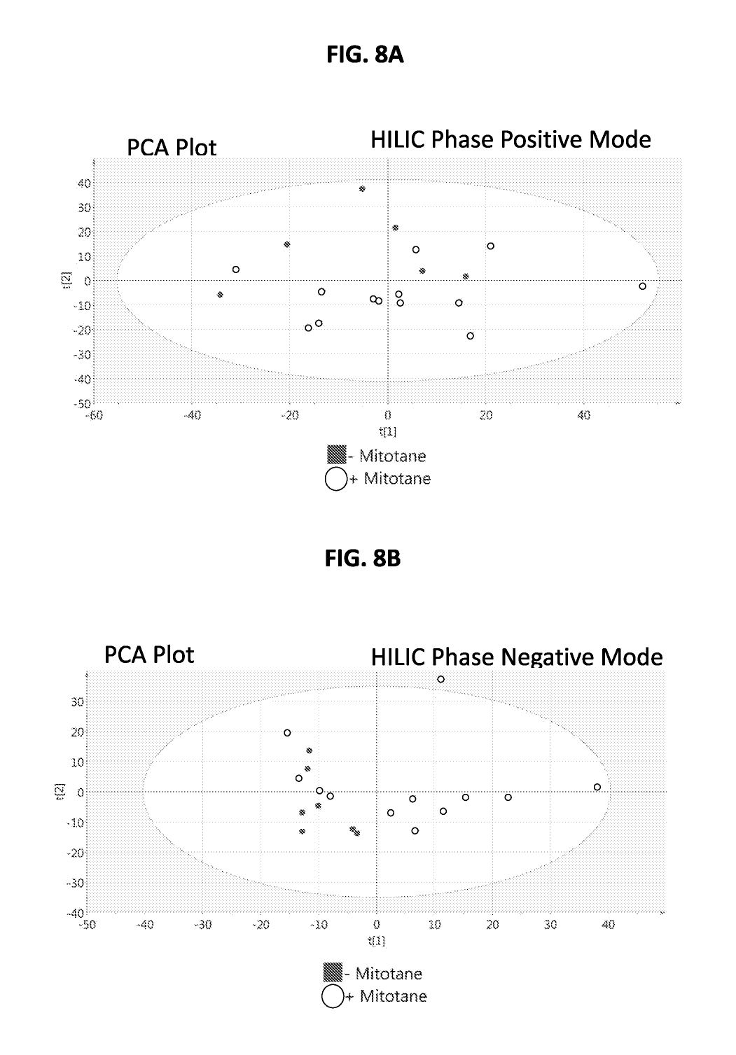

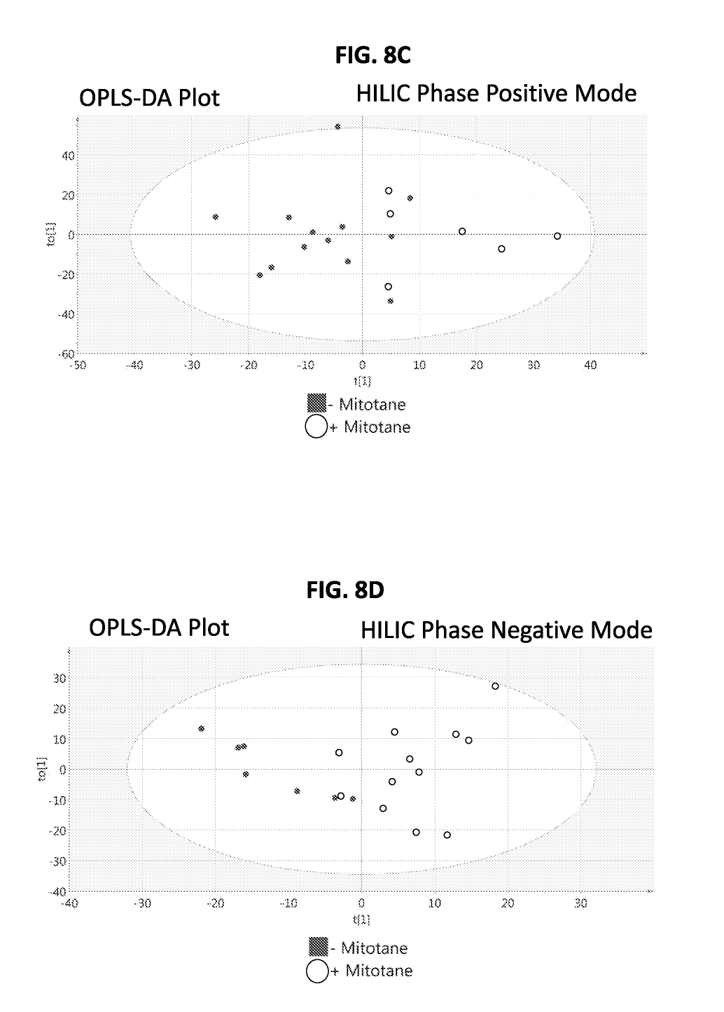

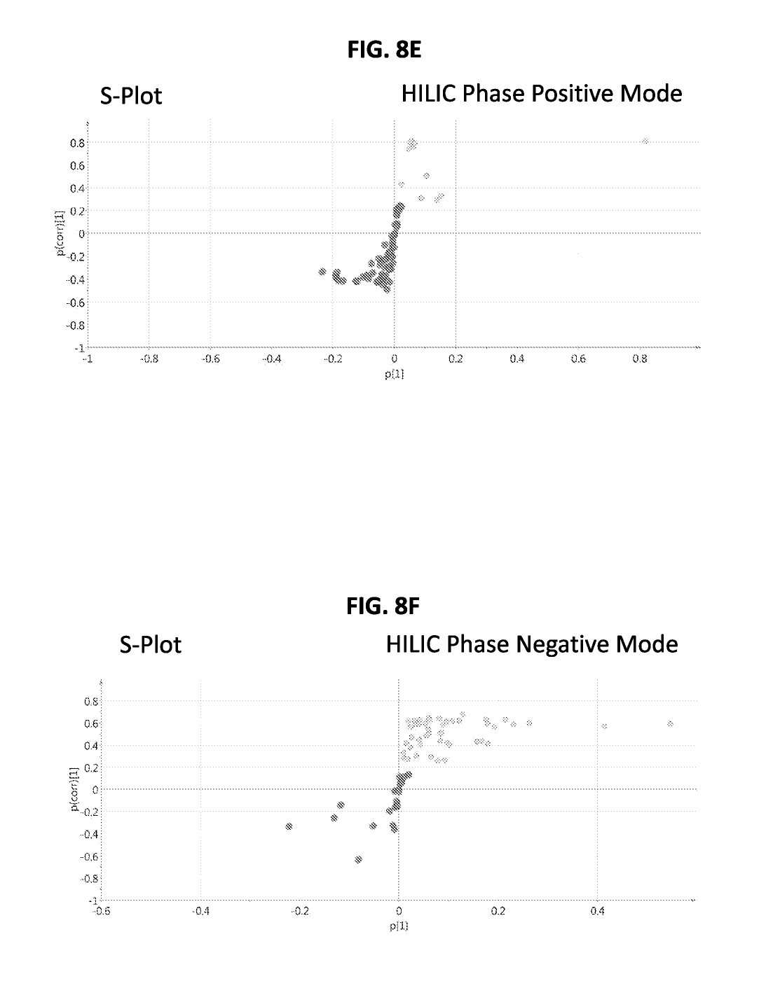

Preoperatively, patients with recurrent and metastatic ACC were treated with mitotane. Given the long half-life (18 to 159 days) and the possibility that metabolites discovered may be mitotane metabolites or related to mitotane metabolism, further analysis of potential drug metabolites was performed. Unsupervised PCA plots show discrimination and separation between patients with and without mitotane (FIGS. 7A, 7B, 8A, and 8B). OPLS-DA plots and S plots showed separation and features correlated with mitotane use, which were excluded from further analysis (FIGS. 7C-7F, and 8C-8F). To verify that these were metabolites of mitotane, the MS/MS fragmentation pattern of a top hit was verified to be a mitotane metabolite.

Metabolites not associated with mitotane ingestion were identified and further studied and the significant features as discovered by different methods are summarized in Table 2.

TABLE 2
Summary Findings of Features.

| | HILIC Phase | | Reverse Phase | |
|---|---|---|---|---|
| | Positive | Negative | Positive | Negative |
| | Mode | Mode | Mode | Mode |
| Total Features | 4507 | 5719 | 6965 | 3582 |
| Progenesis | 74 | 151 | 163 | 68 |
| T-Test, FDR | 34 | 69 | 148 | 50 |
| SIMCA | 17 | 87 | 77 | 10 |
| After ROC Analysis | 74 | 154 | 164 | 89 |
| After Mitotane Analysis | 45 | 15 | 3 | 6 |

Sixty-nine features were identified after exclusion of those associated with mitotane therapy. Specific m/z masses and retention times that are able to differentiate the groups are summarized in Table 3.

TABLE 3
FEATURES IDENTIFIED BY PHASE OF CHROMATOGRAPHY AND IONIZATION.

| | | | | | | AREA |
|---|---|---|---|---|---|---|
| | | | | | | UNDER THE |
| | | | | FOLD | | CURVE |
| | FEATURE | M/Z | RT | CHANGE* | P-VALUE | (AUC) |
| HILIC POSITIVE | FEATURE 1 | 334.186 | 4.88 | 3.92 | <0.0001 | 0.88304 |
| | Creatine riboside | 264.119 | 4.55 | 3.51 | <0.0001 | 0.80702 |
| | Feature 3 | 726.803 | 2.93 | 2.48 (B) | <0.0001 | 0.80936 |
| | Feature 4 | 922.757 | 2.93 | 2.61 (B) | <0.0001 | 0.82105 |
| | Nε,Nε,Nε- | 84.080 | 6.01 | 2.29 (B) | <0.0001 | 0.81053 |
| | trimethyl-L-lysine | | | | | |
| | Feature 6 | 306.153 | 4.97 | 4.20 | <0.0001 | 0.84444 |
| | Feature 7 | 824.780 | 2.93 | 2.51 (B) | <0.0001 | 0.83275 |
| | Nε,Nε,Nε- | 189.159 | 6.01 | 1.86 (B) | <0.0001 | 0.81637 |
| | trimethyl-L-lysine | | | | | |
| | Nε,Nε,Nε- | 144.138 | 6.01 | 1.95 (B) | 0.0001 | 0.89123 |
| | trimethyl-L-lysine | | | | | |
| | Feature 10 | 920.758 | 2.93 | 2.66 (B) | <0.0001 | 0.81754 |
| | Feature 11 | 822.781 | 2.90 | 2.54 (B) | <0.0001 | 0.80234 |
| | Feature 12 | 530.848 | 2.92 | 2.37 (B) | <0.0001 | 0.86667 |
| | Feature 13 | 628.824 | 2.92 | 2.41 (B) | <0.0001 | 0.84912 |
| | Feature 14 | 626.826 | 2.92 | 2.39 (B) | <0.0001 | 0.82339 |
| | Feature 15 | 528.850 | 2.92 | 2.35 (B) | <0.0001 | 0.82807 |
| | Feature 16 | 630.824 | 2.92 | 2.41 (B) | <0.0001 | 0.86784 |
| | Feature 17 | 332.895 | 2.92 | 2.26 (B) | <0.0001 | 0.82456 |
| | Feature 18 | 532.847 | 2.92 | 2.41 (B) | <0.0001 | 0.84912 |
| | Feature 19 | 189.048 | 6.01 | 2.04 (B) | 0.0004 | 0.85146 |
| | Feature 20 | 724.804 | 2.92 | 2.48 (B) | <0.0001 | 0.80234 |
| | Feature 21 | 146.981 | 5.24 | 2.30 (B) | 0.0002 | 0.84211 |
| | Feature 22 | 234.917 | 2.92 | 2.21 (B) | <0.0001 | 0.86199 |
| | Feature 23 | 236.915 | 2.92 | 2.24 (B) | <0.0001 | 0.83392 |
| | Feature 24 | 172.084 | 2.63 | 4.27 | <0.0001 | 0.80702 |
| | Feature 25 | 432.870 | 2.89 | 2.36 (B) | <0.0001 | 0.87135 |
| | Feature 26 | 136.940 | 2.92 | 2.15 (B) | <0.0001 | 0.81871 |
| | Feature 27 | 318.191 | 4.47 | 2.38 | 0.0001 | 0.85497 |
| | Feature 28 | 430.872 | 2.88 | 2.35 (B) | <0.0001 | 0.88187 |
| | Feature 29 | 126.102 | 4.27 | 3.05 (B) | 0.0001 | 0.83158 |
| | Creatine riboside | 264.119 | 4.35 | 2.63 | <0.0001 | 0.8 |
| | Feature 31 | 336.892 | 2.92 | 2.30 (B) | <0.0001 | 0.80351 |
| | Feature 32 | 105.954 | 5.23 | 2.15 (B) | 0.0002 | 0.84327 |
| | Feature 33 | 103.955 | 5.23 | 2.11 (B) | 0.0003 | 0.84561 |
| | Feature 34 | 138.938 | 2.92 | 2.17 (B) | <0.0001 | 0.86433 |
| | Feature 35 | 271.104 | 5.20 | 2.83 (B) | <0.0001 | 0.86316 |
| | Feature 36 | 188.071 | 2.39 | 2.38 (B) | 0.0001 | 0.85965 |
| | Feature 37 | 302.159 | 4.69 | 1.41 | 0.0378 | 0.8655 |
| | Feature 38 | 219.042 | 0.78 | 2.25 (B) | 0.0001 | 0.8655 |
| | Feature 39 | 156.017 | 3.99 | 3.33 (B) | 0.0001 | 0.85731 |
| | Feature 40 | 141.138 | 6.01 | 1.72 (B) | 0.0004 | 0.86784 |
| | Feature 41 | 153.067 | 4.27 | 2.80 (B) | 0.0011 | 0.88304 |
| | Feature 42 | 339.099 | 4.66 | 2.42 | 0.0001 | 0.87485 |
| | Feature 43 | 134.963 | 0.78 | 2.76 (B) | 0.0003 | 0.88538 |
| | Feature 44 | 384.119 | 5.02 | 1.52 | <0.0001 | 0.8655 |
| | Feature 45 | 238.946 | 0.79 | 2.05 (B) | 0.0061 | 0.87719 |
| HILIC Negative | Feature 1 | 499.088 | 0.37 | 3.40 (B) | <0.0001 | 0.81 |
| | Feature 2 | 461.115 | 0.37 | 3.68 (B) | <0.0001 | 0.83 |
| | Feature 3 | 225.114 | 0.44 | 7.38 (B) | <0.0001 | 0.82 |
| | Feature 4 | 220.97 | 0.46 | 3.49 (B) | <0.0001 | 0.85 |
| | Feature 5 | 209.949 | 0.52 | 6.51 (B) | <0.0001 | 0.84 |
| | Feature 6 | 160.076 | 0.70 | 10.83 (B) | 0.0002 | 0.79 |
| | Feature 7 | 103.040 | 2.20 | 2.97 (B) | <0.0001 | 0.89 |
| | L-tryptophan | 203.082 | 2.21 | 2.96 (B) | <0.0001 | 0.85 |
| | 3-methylhistidine | 151.051 | 4.27 | 4.05 (B) | <0.0001 | 0.82 |
| | 3-methylhistidine | 168.077 | 4.27 | 3.91 (B) | <0.0001 | 0.84 |
| | Feature 11 | 396.023 | 4.63 | 2.34 | 0.0003 | 0.78 |
| | Feature 12 | 351.056 | 4.91 | 4.93 | <0.0001 | 0.82 |
| | Feature 13 | 209.030 | 5.50 | 2.47 | 0.0281 | 0.67 |
| | Feature 14 | 145.098 | 6.13 | 5.70 (B) | <0.0001 | 0.84 |
| | Feature 15 | 625.184 | 6.52 | 1.51 | 0.0001 | 0.79 |
| RP Positive | Feature 1 | 186.148 | 3.15 | 2.80 | 0.0007 | 0.76 |
| | Feature 2 | 93.071 | 4.22 | 2.50 | 0.0076 | 0.71 |
| | Feature 3 | 118.033 | 6.03 | 1.76 | 0.0005 | 0.77 |
| RP Negative | Feature 1 | 411.127 | 3.46 | 1.11 | 0.0182 | 0.69 |
| | Feature 2 | 427.231 | 4.07 | 4.81 | 0.0002 | 0.79 |
| | Feature 3 | 181.134 | 4.16 | 3.29 | 0.0001 | 0.80 |
| | Feature 4 | 276.089 | 5.00 | 1.64 | 0.0018 | 0.74 |
| | Feature 5 | 499.198 | 5.07 | 5.66 | 0.0006 | 0.76 |
| | Feature 6 | 106.066 | 5.47 | 1.63 | <0.0001 | 0.81 |

*Fold change of malignant compared to benign, except when noted by a (B).

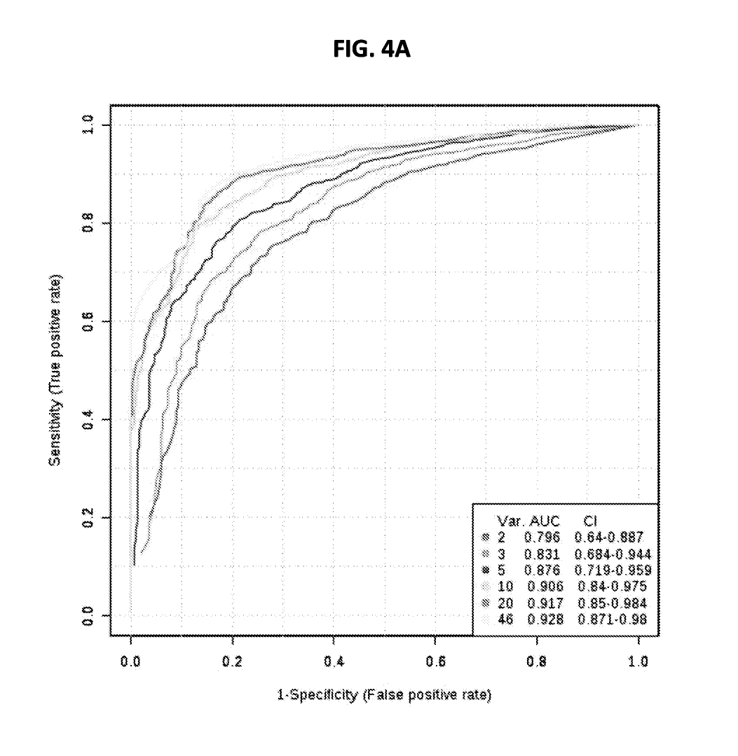

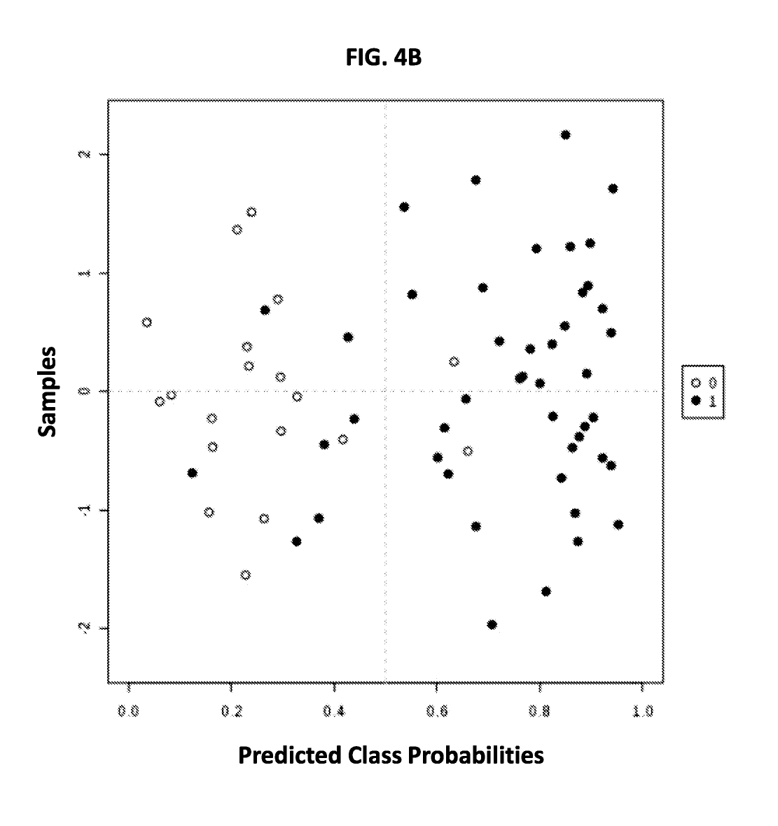

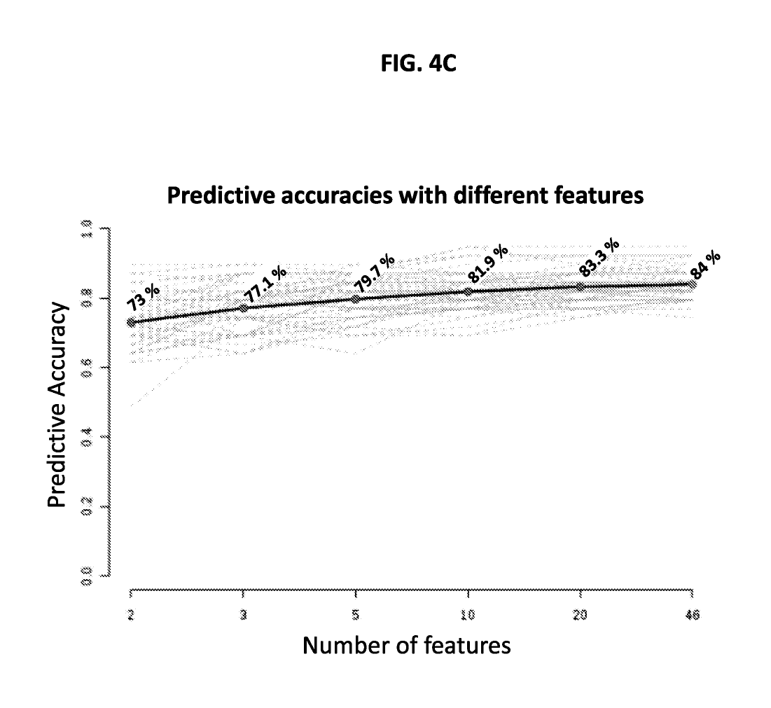

The significant features of parent compounds were combined in a multivariate random forest model to classify benign and malignant adrenal tumors. Of the 69 features, 46 features were parent compounds and not related to mitotane ingestion. A combination of the 46 features included in the model showed that the AUC could be improved to 0.928 with the inclusion of all features (FIG. 4A). The predicted accuracy of classification by urinary metabolic analysis utilizing these features was 84% (FIG. 4B).

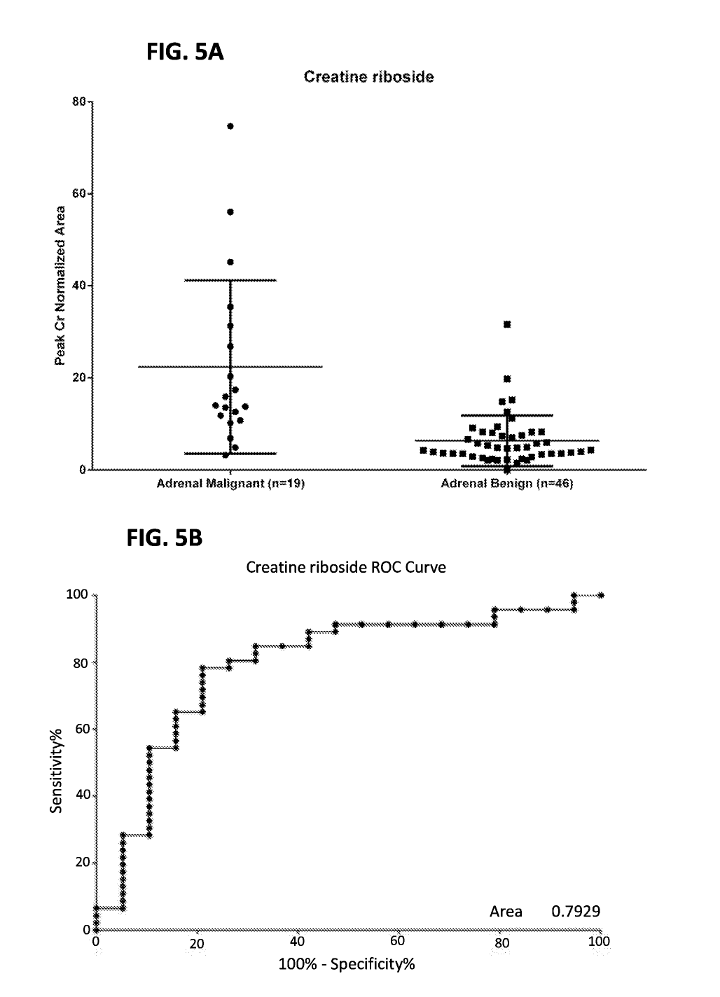

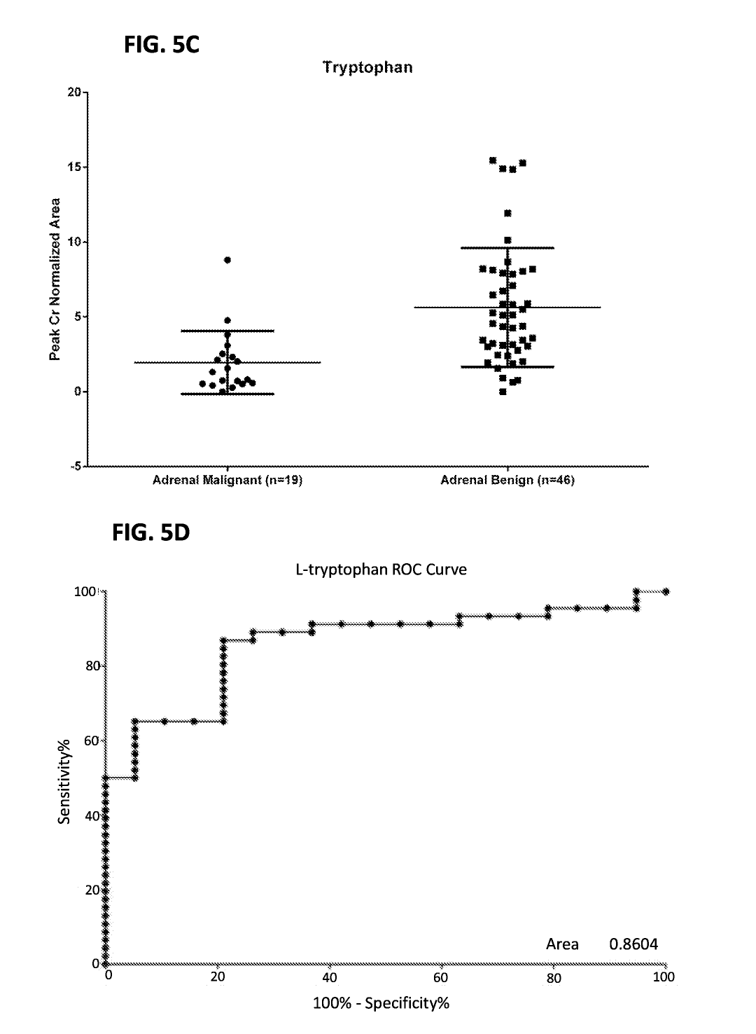

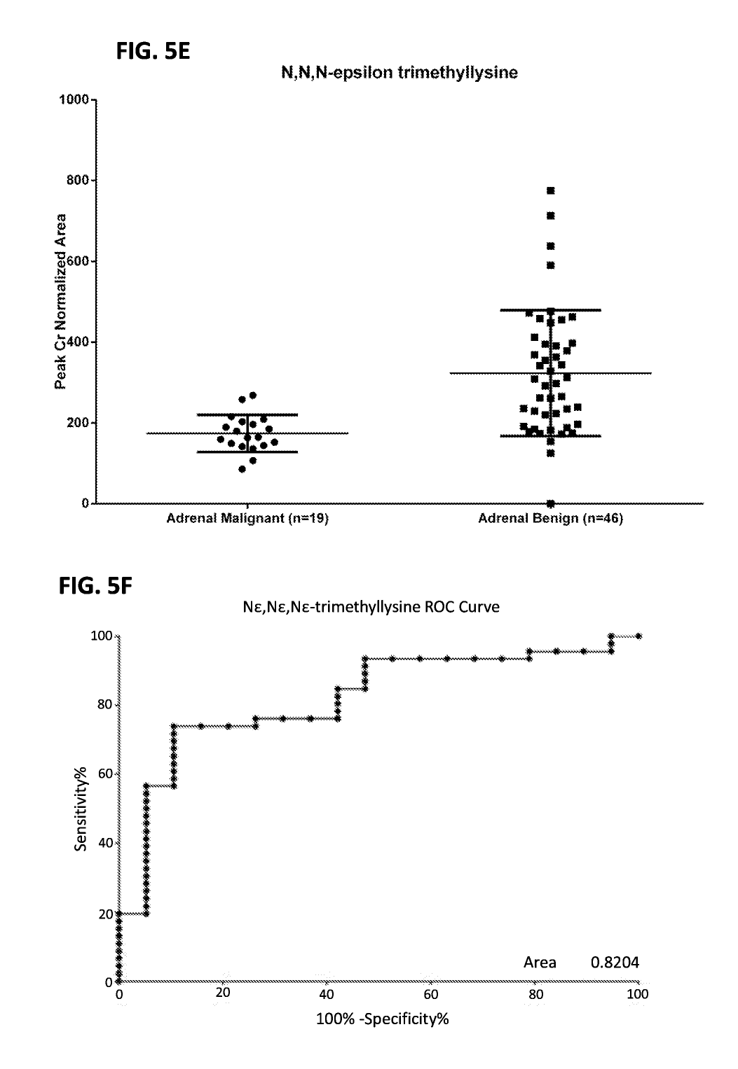

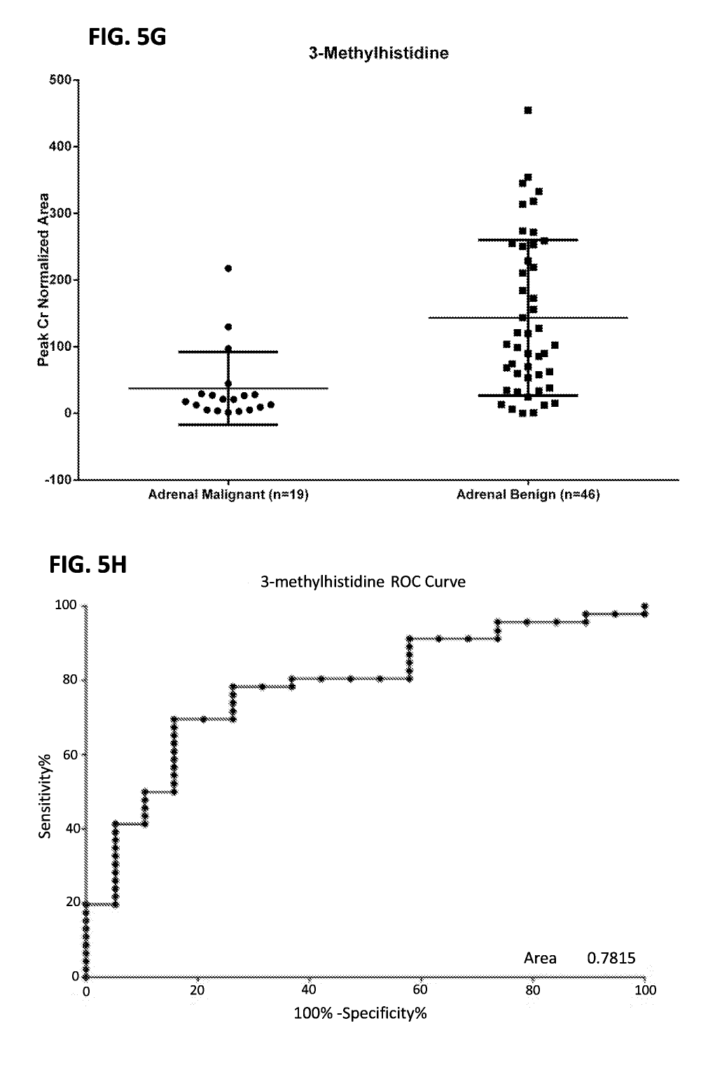

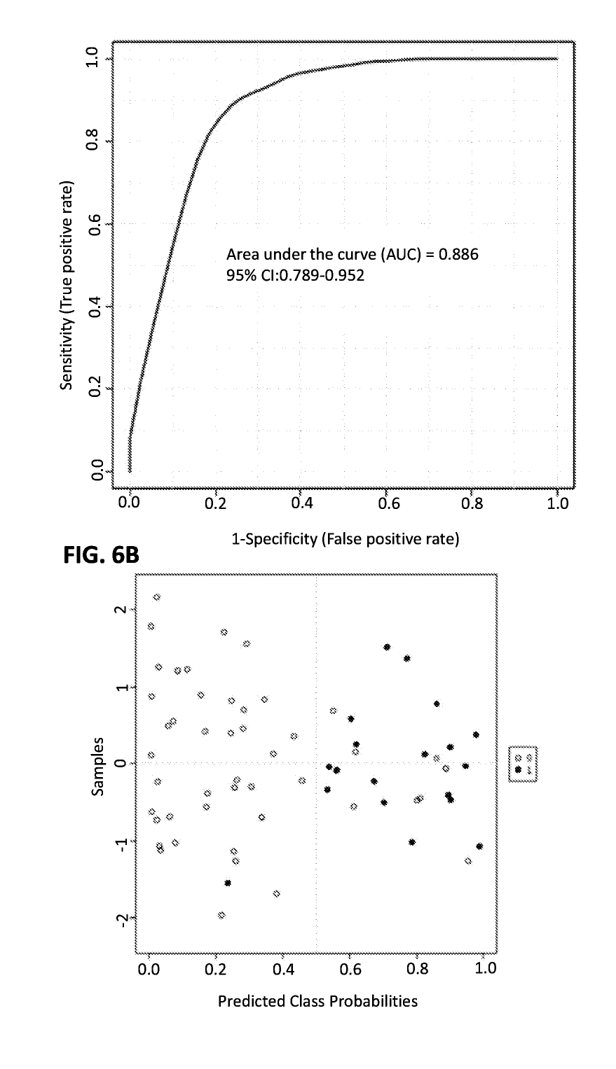

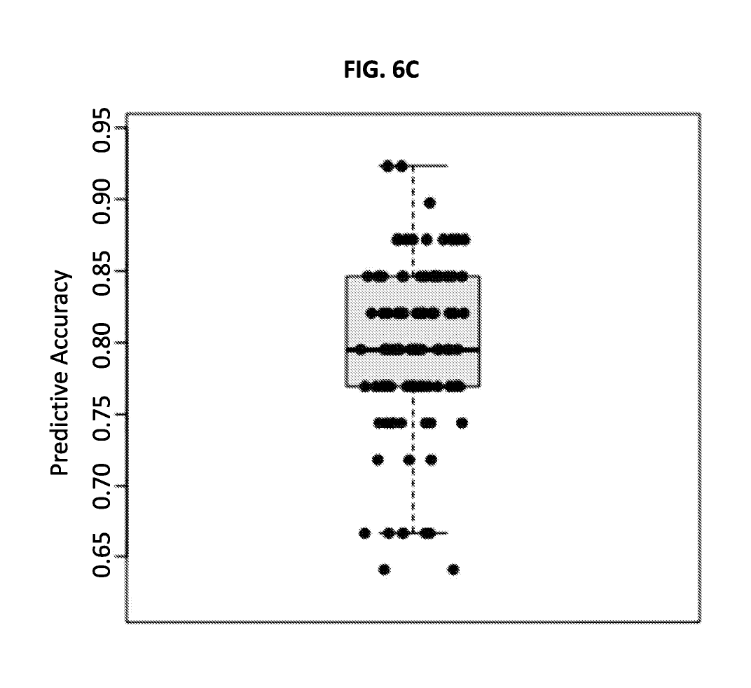

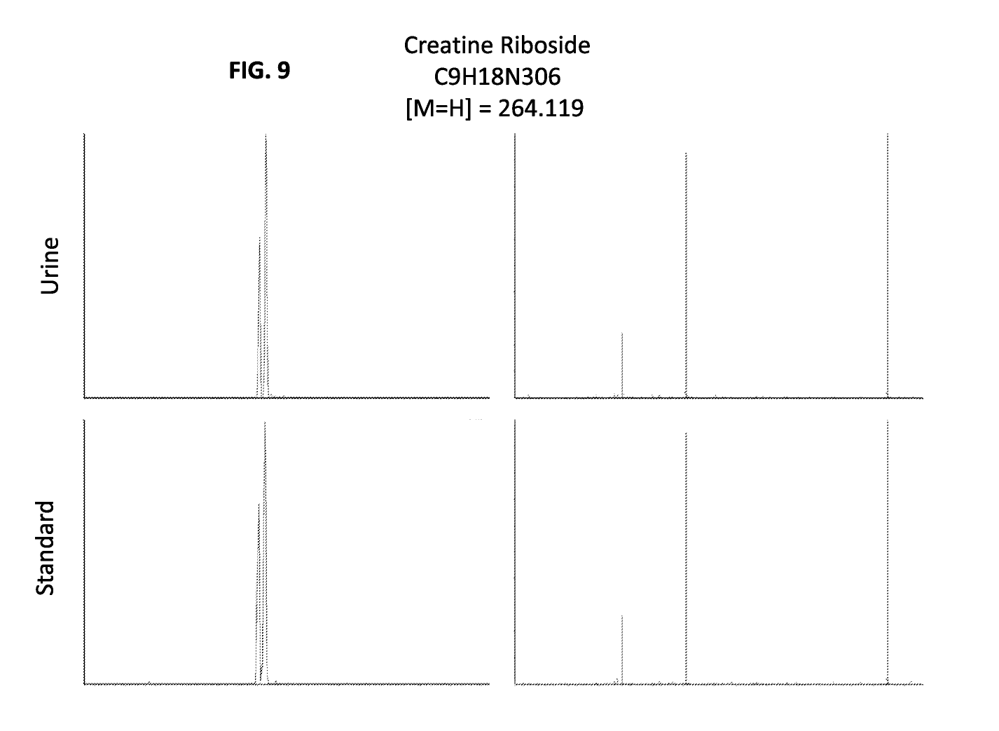

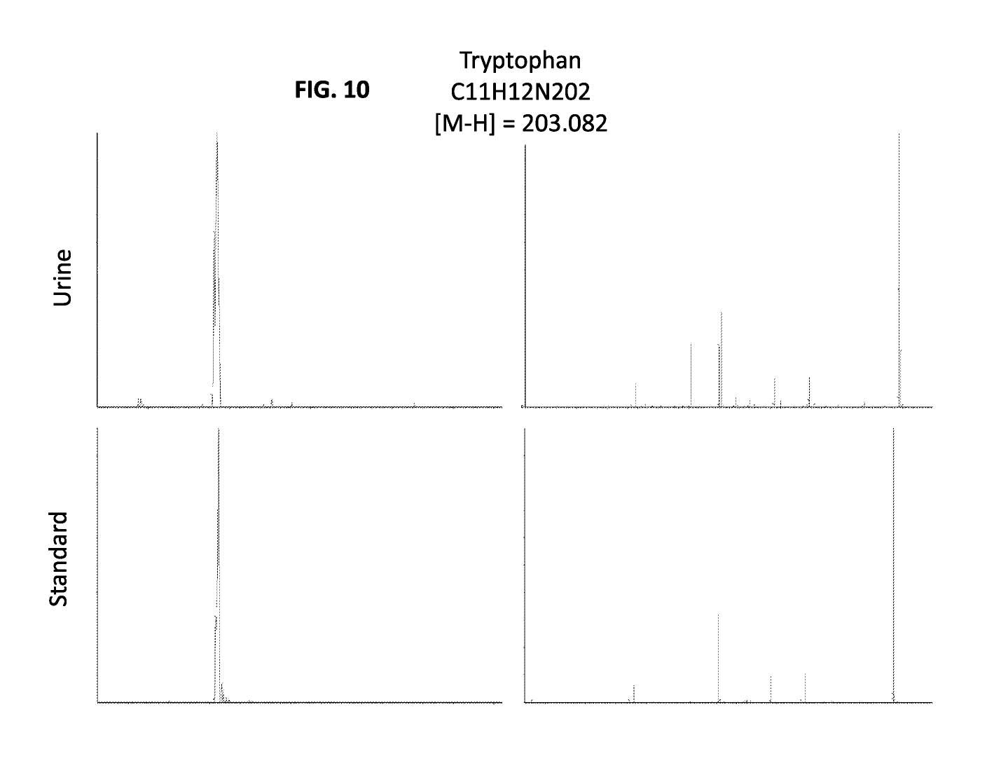

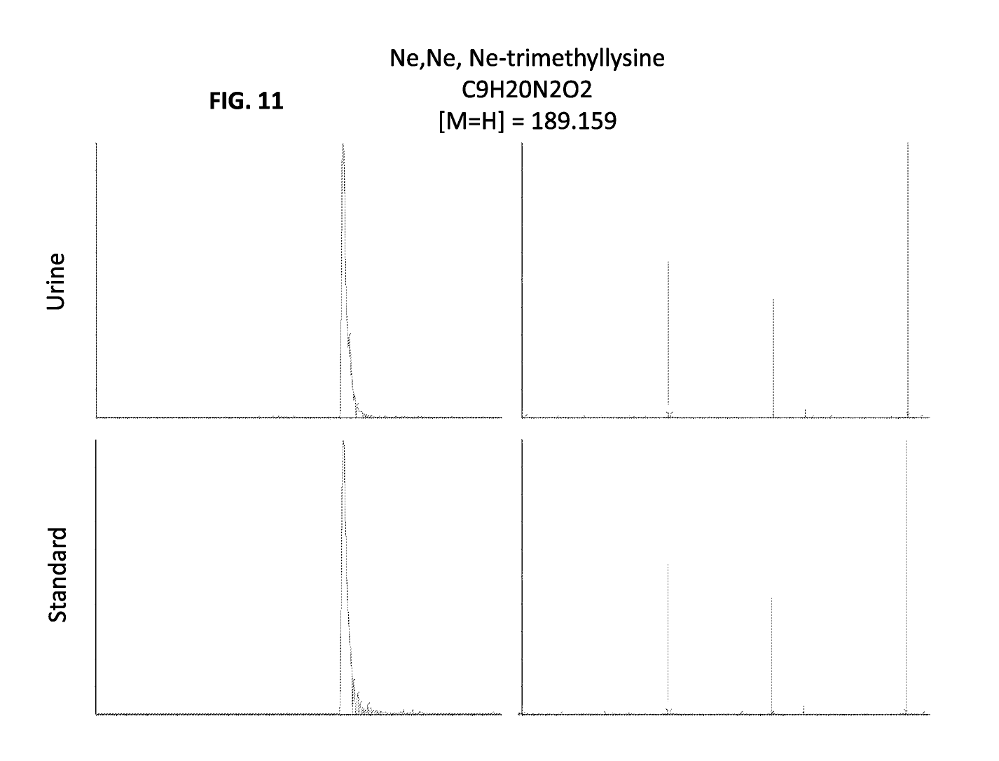

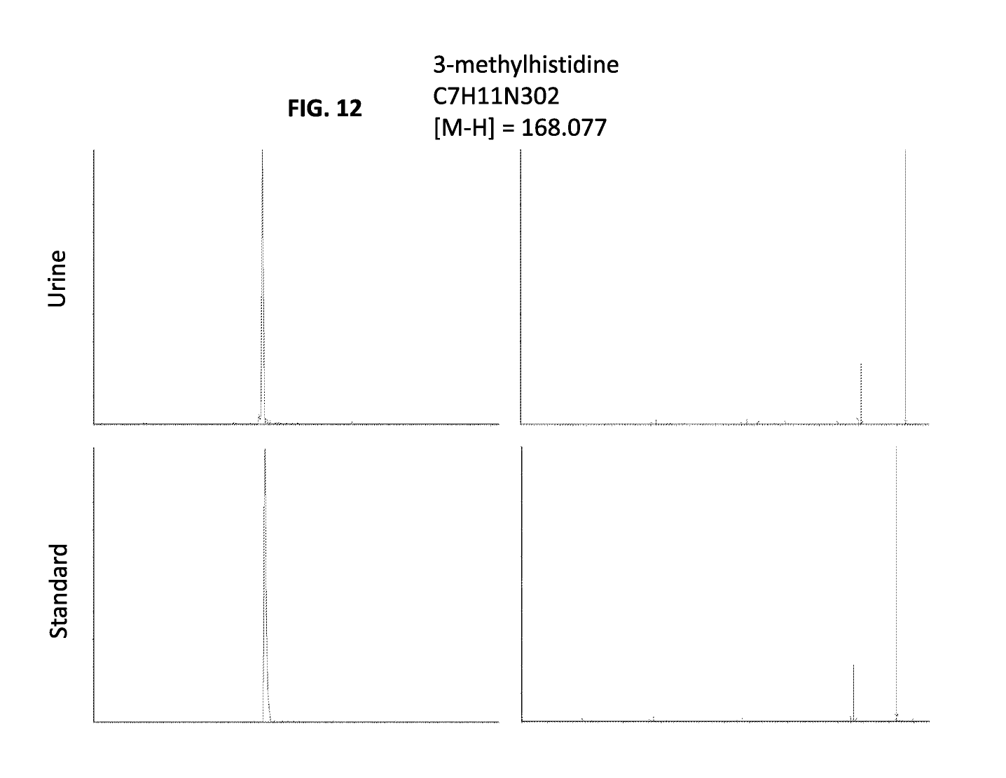

Among the 69 features identified, four features were identified through MS/MS fragmentation and online metabolomics database searches. The four metabolites identified through fragmentation patterns and database searches were creatine riboside, Nε,Nε,Nε-trimethyl-L-lysine, 3-methylhistidine, and L-tryptophan (FIGS. 9, 10, 11, and 12). These four metabolites were quantitated on an independent platform. Creatine riboside was elevated over two-fold in patients with ACC compared to patients with benign adrenal tumors (FIG. 5A, 2.1 fold change, p=0.0001). Receiver operator characteristic curve showed an area AUC of 0.793 (FIG. 5B). L-Tryptophan, (3.0 fold change, p<0.0001),Nε,Nε,Nε-trimethyl-L-lysine(1.8 fold change, p<0.0001), and 3-methylhistidine (3.0 fold change, p=0.0003) were each lower in patients with ACC compared to patients with benign adrenal tumors (FIGS. 5C, 5E, and 5G). Receiver operator characteristic curves showed an AUC of 0.860, 0.820, and 0.782, respectively (FIGS. 5D, 5F, and 5H). Using the four identified metabolites as a panel, the AUC was 0.89 (FIG. 6A). Use of the four features further showed a sensitivity of 94.7% and specificity of 82.6%. The positive predictive value was 69.2% and negative predictive value was 97.4%. The model was predicted to be 79.5% accurate in diagnosing malignancy when using a cross validation model (FIG. 6C).

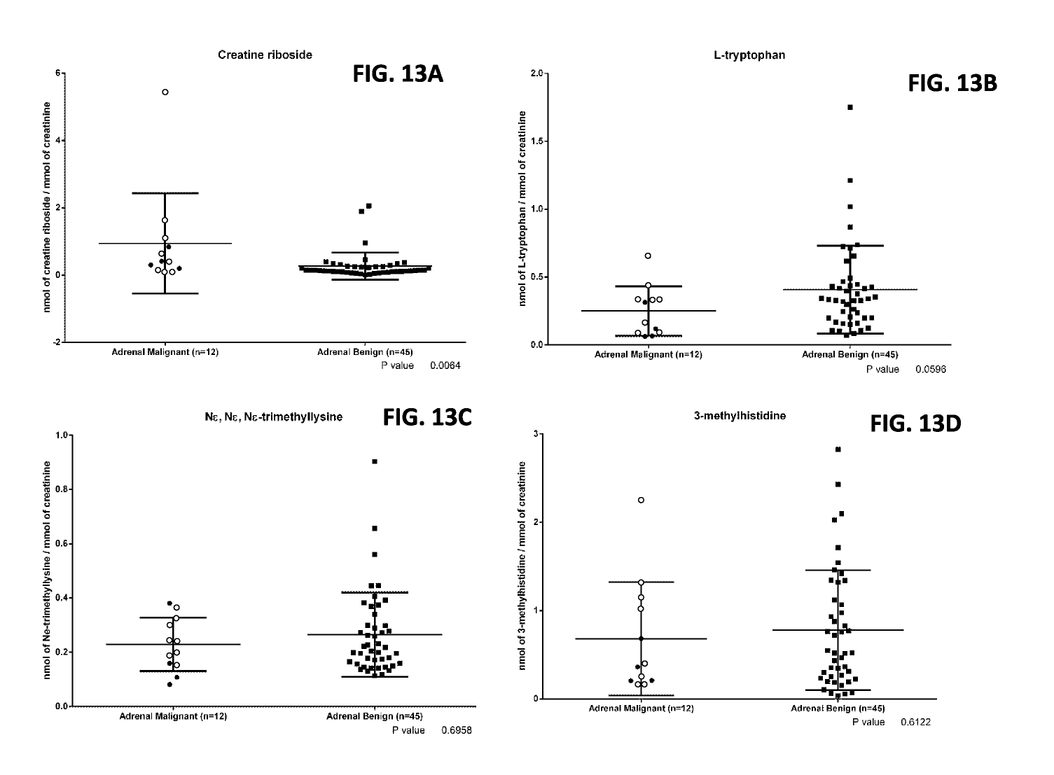

The four identified metabolites were analyzed in an independent set of urine samples consisting of twelve patients with ACC and forty-five patients with benign adrenal tumors (Tables 1A, 1C). Creatine riboside was validated and continued to show a significant difference between the two groups (FIG. 13A). L-Tryptophan, Nε,Nε,Nε-trimethyl-L-lysine, and 3-methylhistidine were found to be not significantly different (FIGS. 13B, 13C, and 13D). Since these three metabolites could be affected by diet and that eight of the twelve patients were not fasting at the time of urine collection, fasting status was compared. All of the metabolites were affected by diet except creatine riboside. Given the rarity of ACC and the effect of diet, the cohorts were combined without the non-fasting patients. All four metabolites were significantly different with both cohorts combined.

### Discussion

This Example discloses the first untargeted metabolomics examination of patients with benign adrenal tumors and ACC. An unbiased examination of the data shows that the two groups can be discriminated by a urinary metabolomics approach. Analysis of specific features identified that patients with ACC had higher levels of creatine riboside and lower levels of L-tryptophan, Nε,Nε,Nε-trimethyl-L-lysine and 3-methylhistidine when compared to patients with benign adrenal tumors. Furthermore, the combination of the four identified markers is superior to one marker, and can be used in the diagnosis of ACC.

Creatine riboside, which was significantly elevated in patients with ACC. In addition to elevated levels in the urine, increased creatine riboside was found in the tumors of patients with lung cancer compared to normal adjacent tissue. Creatine riboside, therefore, may increase the index of suspicion of a potential malignant neoplasm. Although the function of creatine riboside has yet to be elucidated, the fact that both lung and adrenal cancer have elevated levels may indicate that this metabolite is involved in tumorigenesis.

The lower levels of L-tryptophan may be related to degradation of tryptophan by the adrenal tumor as a way to evade the body's antitumor response. Tryptophan is degraded to the metabolite kynurenine in glioma cell lines by the enzyme tryptophan-2,3-dioxygenase (TDO) and this results in immune evasion by the tumor. Although the data in this study supports global tryptophan degradation, kynurenine was not elevated in patients with ACC. This may be explained by the fact that kynurenine may be further degraded or potentially taken up by the ACC cells through the aryl hydrocarbon receptor. This evasion of the immune system would explain the relative lack of tumor infiltrating lymphocytes in ACC on histological examination. Further studies will determine if the enzymes involved in tryptophan degradation such as TDO and indoleamine 2,3-dioxygenase (IDO1) are involved in ACC and evasion of the immune response.

The third metabolite identified, Nε,Nε,Nε-trimethyl-L-lysine, is associated with trimethylation of lysine residues on histone complexes. Histone lysine methylation is critical in the regulation of gene expression, cell cycle, genome stability and nuclear architecture. The present data showed that the metabolite Nε,Nε,Nε-trimethyl-L-lysine, was decreased in urine of patients with ACC. Immunohistochemistry studies revealed that a decrease in trimethylation of lysine 27 at histone H3 was associated with poor prognosis in patients with breast cancer regardless of estrogen receptor (ER) status. When the patients were stratified by EZH2, the methyltransferase responsible for trimethylation of lysine 27, and trimethylation status, patients with high EZH2 and low trimethylation expression had the worst prognosis. Currently there is high interest in targeting EZH2 and other associated methyltransferases given their strong implication in oncologic dysregulation. The decreased levels of Nε,Nε,Nε-trimethyl-L-lysine may indicate that EZH2 expression is decreased in patients with ACC, with subsequent activation of genes found at histone H3. However, since Nε,Nε,Nε-trimethyl-L-lysine may also be found in myosin, and thus a potential alternative explanation for the decreased Nε,Nε,Nε-trimethyl-L-lysine may be inhibition of muscle breakdown by ACC tumors.

3-Methylhistidine, like Nε,Nε,Nε-trimethyl-L-lysine, is also found in muscle and is a potential diagnostic biomarker of ACC. 3-Methylhistidine was initially identified and associated with the actin component of muscle. 3-Methylhistidine is not further metabolized by the human body and is excreted primarily in the urine. Furthermore, 3-methylhistidine urinary excretion comes primarily from muscle breakdown and the methylated amino acid has been used as a surrogate of whole body protein breakdown in patients receiving total parental nutrition, and with trauma and sepsis. 3-Methylhistidine, therefore, would be expected to be elevated in patients with ACC due to whole-body protein breakdown, malnutrition, or sarcopenia. However, the data presented in this study shows that 3-methylhistidine is higher in patients with benign adrenal tumors compared to patients with ACC. This is an unexpected result. Given that Nε,Nε,Nε-trimethyl-L-lysine and 3-methylhistidine are both decreased in patients with ACC and both may be involved in muscle breakdown, this may indicate that ACC tumors are secreting a factor that reduces the breakdown of muscle. This factor may be the growth factor IGF2. ACC tumors overexpress IGF2 and IGF2 is involved in muscle growth and development. Overexpression and secretion of IGF2 may result in decreased muscle breakdown, and therefore decreased metabolites of muscle breakdown in the urine of patients with ACC.

To summarize this study, urine samples from patients with benign adrenal tumors and ACC were analyzed using a metabolomic approach in an untargeted unbiased fashion. Sixty-seven features were identified that could differentiate these two groups of patients. Furthermore, four cancer-related metabolites, creatine riboside, L-tryptophan, Nε,Nε,Nε-trimethyl-L-lysine, and 3-methylhistidine were identified and validated on an independent platform through quantitation.

### Example 3

### Method to Treat ACC

This example describes a particular method that can be used to treat ACC in humans by administration of one or more agents that alter one or more of the disclosed metabolites with altered levels in ACC samples. Although particular methods, dosages, and modes of administrations are provided, one skilled in the art will appreciate that variations can be made without substantially affecting the treatment.

Based upon the teaching disclosed herein, ACC can be treated by administering a therapeutically effective amount of an agent that decreases the level of creatine riboside, and/or increases the level of L-tryptophan, Nε,Nε,Nε-trimethyl-L-lysine, and/or 3-methylhistidine, thereby reducing one or more signs or symptoms associated with the ACC.

Briefly, the method can include screening subjects to determine if they have a ACC. Subjects having ACC are selected. In one example, subjects having increased levels of creatine riboside and decreased levels of L-tryptophan, Nε,Nε,Nε-trimethyl-L-lysine, and 3-methylhistidine as compared to reference values (indicative of levels of the four aforementioned metabolites in a subject with a benign adrenocortical tumor) are selected. In one example, a clinical trial would include half of the subjects following the established protocol for treatment of ACC (such as a normal chemotherapy/radiotherapy/surgery regimen). The other half would follow the established protocol for treatment of the ACC (such as a normal chemotherapy/radiotherapy/surgery regimen) in combination with administration of the therapeutic compositions described above. In some examples, the tumor is surgically excised (in whole or part) prior to treatment with the therapeutic compositions. In another example, a clinical trial would include half of the subjects following the established protocol for treatment of ACC (such as a normal chemotherapy/radiotherapy/surgery regimen). The other half would follow the administration of the therapeutic compositions described above. In some examples, the tumor is surgically excised (in whole or part) prior to treatment with the therapeutic compositions.

### Screening Subjects

In some examples, the subject is first screened to determine if they have ACC. In particular examples, the subject is screened to determine if the adrenocortical tumor is malignant, indicating ACC, or benign by obtaining a urine sample from the subject and analyzing it for the presence of creatine riboside, L-tryptophan, Nε,Nε,Nε-trimethyl-L-lysine, and 3-methylhistidine, wherein the presence of a 2-fold increase in creatine riboside and at least a 1.5-decrease in levels of L-tryptophan, Nε,Nε,Nε-trimethyl-L-lysine, and 3-methylhistidine indicates that the tumor is malignant and further that it can be treated with the disclosed therapies. However, such pre-screening is not required prior to administration of the therapeutic compositions disclosed herein (such as those that include an agent that decreases levels of creatine riboside and/or increases levels of L-tryptophan, Nε,Nε,Nε-trimethyl-L-lysine, and/or 3-methylhistidine).

### Pre-Treatment of Subjects

In particular examples, the subject is treated prior to administration of a therapeutic composition that includes one or more agents to one or more of the disclosed urinary metabolites altered in a malignant adrenocortical tumor. However, such pre-treatment is not always required, and can be determined by a skilled clinician. For example, the tumor can be surgically excised (in total or in part) prior to administration of the therapy. In addition, the subject can be treated with an established protocol for treatment of the particular tumor present (such as a normal chemotherapy/radiotherapy regimen).

### Administration of Therapeutic Compositions

Following subject selection, a therapeutic effective dose of the composition is administered to the subject, wherein the composition includes one or more agents capable of decreasing levels of creatine riboside and/or increasing levels of L-tryptophan, Nε,Nε,Nε-trimethyl-L-lysine, and 3-methylhistidine. In some examples, the therapeutic dose is administrated by intravenous or oral administration. Also for metabolites that are at decreased levels in a subject with a malignant adrenocortical tumor (e.g., L-tryptophan, Nε,Nε,Nε-trimethyl-L-lysine, and 3-methylhistidine), such metabolites or combinations of the three metabolites can be administered. Administration of the therapeutic compositions can be continued after chemotherapy and radiation therapy is stopped and can be taken long term (for example over a period of months or years).

### Assessment

Following the administration of one or more therapies, subjects having a malignant tumor (for example ACC) can be monitored for tumor treatment, such as regression or reduction in metastatic lesions, tumor growth or vascularization. In particular examples, subjects are analyzed one or more times, starting 7 days following treatment. Subjects can be monitored using any method known in the art. For example, diagnostic imaging can be used (such as x-rays, CT scans, MRIs, ultrasound, fiber optic examination, and laparoscopic examination), as well as analysis of biological samples from the subject (for example analysis of urine, tissue biopsy, or other biological samples), such as analysis of the type of cells present, or analysis for a particular tumor marker. In one example, if the subject has advanced ACC, assessment can be made using a non-invasive method including a urinary sample. It is also contemplated that subjects can be monitored for the response of their tumor(s) to therapy during therapeutic treatment by at least the aforementioned methods.

### Additional Treatments

In particular examples, if subjects are stable or have a minor, mixed or partial response to treatment, they can be re-treated after re-evaluation with the same schedule and preparation of agents that they previously received for the desired amount of time, such as up to a year of total therapy. A partial response is a reduction in size or growth of some tumors, but an increase in others.

### Example 4

### Diagnosis of a Malignant Adrenocortical Tumor

This example describes particular methods that can be used to diagnose or prognose a malignant adrenocortical tumor in a subject, such as ACC in a human. However, one skilled in the art will appreciate that similar methods can be used. In some examples, such diagnosis is performed before treating the subject (for example as described in Example 3).

A urine sample is obtained from a subject suspected of having a malignant adrenocortical tumor. Urine samples are deproteinated by methods detailed in Example 1 and metabolites (i.e., 3-methylhistidine, Nε,Nε,Nε-trimethyl-L-lysine, L-tryptophan, and creatine riboside) in the deproteinated samples are quantified by ELISA, mass spectrophotometry or nuclear magnetic resonance. Detection of an at least 2-fold increase in creatine riboside, an at least a 1.8-fold decrease in Nε,Nε,Nε-trimethyl-L-lysine, and an at least 3-fold decrease in L-tryptophan and 3-methylhistidine relative to control values (e.g., levels of the given metabolites in a benign adrenocortical tumor or a reference value known to be indicative of such levels in a benign adrenocortical tumor) is indicative that the subject has a malignant adrenocortical tumor.

The results of the test are provided to a user (such as a clinician or other health care worker, laboratory personnel, or patient) in a perceivable output that provides information about the results of the test. The output is a graphical output showing a cut-off value or level that indicates the presence of a malignant adrenocortical tumor. The output is communicated to the user, for example by providing an output via physical, audible, or electronic means (for example by mail, telephone, facsimile transmission, email, or communication to an electronic medical record). The output is accompanied by guidelines for interpreting the data, for example, numerical or other limits that indicate the presence or absence of metastasis. The guidelines need not specify whether metastasis is present or absent, although it may include such a diagnosis. The indicia in the output can, for example, include normal or abnormal ranges or a cutoff, which the recipient of the output may then use to interpret the results, for example, to arrive at a diagnosis, prognosis, or treatment plan. Based upon the results, a therapeutic regimen is or is not recommended.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of distinguishing a benign adrenocortical tumor from a malignant adrenocortical tumor, comprising:
measuring creatine riboside, L-tryptophan, Nε,Nε,Nε-trimethyl-L-lysine, and 3-methylhistidine in a biological sample obtained from a subject with an adrenocortical tumor; and
identifying an increase in creatine riboside and a decrease in L-tryptophan, Nε,Nε,Nε-trimethyl-L-lysine, and 3-methylhistidine in the biological sample from a subject with an adrenocortical tumor when compared to a control indicates a malignant adrenocortical tumor.

2. The method of claim 1, wherein an increase in creatine riboside is an at least a two-fold increase.

3. The method of claim 1, wherein a decrease in L-tryptophan and 3-methylhistidine is at least a 3-fold decrease.

4. The method of claim 1, wherein a decrease in Nε,Nε,Nε-trimethyl-L-lysine is at least a 1.8-fold decrease.

5. The method of claim 1, wherein the method is used for diagnosing or prognosing a subject with adrenocortical carcinoma.

6. The method of claim 1, wherein the control is a benign adrenocortical tumor or a set of reference values representative of the levels of creatine riboside, L-tryptophan, Nε,Nε,Nε-trimethyl-L-lysine and 3-methylhistidine in a subject with a benign adrenocortical tumor.

7. The method of claim 1, wherein the biological sample is a urine sample.

8. The method of claim 1, wherein measuring is performed by using liquid chromatography-mass spectrometry (LCMS,) enzyme linked immunosorbent assay (ELISA), chemoluminiscence- or fluorescence-based assay.

9. The method of claim 1, further comprising obtaining the biological sample from the subject with the adrenocortical tumor.

10. A method of treating a malignant adrenocortical tumor in a subject, comprising:
administering to the subject an effective amount of an agent that alters an creatine riboside, L-tryptophan, Nε,Nε,Nε-trimethyl-L-lysine and/or 3-methylhistidine, thereby treating the malignant adrenocortical tumor.

11. The method of claim 10, wherein the agent decreases creatine riboside and/or increases L-tryptophan, Nε,Nε,Nε-trimethyl-L-lysine and/or 3-methylhistidine.

12. The method of claim 10, wherein the method is used for treating a subject with adrenocortical carcinoma.

13. A method of determining the effectiveness of an agent for the treatment of a malignant adrenocortical tumor in a subject with the malignant adrenocortical tumor, comprising:
detecting an creatine riboside, L-tryptophan, Nε,Nε,Nε-trimethyl-L-lysine and 3-methylhistidine in a biological sample from the subject following treatment with the agent;
wherein a decrease in creatine riboside and an increase in L-tryptophan, Nε,Nε,Nε-trimethyl-L-lysine and 3-methylhistidine levels following treatment as compared to a reference value for each, indicates that the agent is effective for the treatment of the malignant adrenocortical tumor in the subject.

14. The method of claim 13, wherein the reference value represents level of creatine riboside, L-tryptophan, Nε,Nε,Nε-trimethyl-L-lysine and 3-methylhistidine in a sample from the subject prior to treatment with the agent.

15. A device for characterizing an adrenocortical tumor, comprising:
a surface for collecting one or more urinary metabolites associated with a malignant adrenocortical tumor, wherein the one or more urinary metabolites include creatine riboside, Nε,Nε,Nε-trimethyl-L-lysine, L-tryptophan, and/or 3-methylhistidine; and
one or more detecting molecules capable of binding to the one or more urinary metabolites of interest at an addressable location and generating a complex product for each detected urinary metabolite.

16. The device of claim 15, wherein the device is a lateral flow device.

17. The device of claim 16, wherein the lateral flow device is a dip stick configuration.

18. The device of claim 15, further comprising a readout area in which the generated complex product for each detected urinary metabolite is displayed and can be quantitated.
